(19) United States
(12) Patent Application Publication
(10) Pub. No.: US 2010/0297627 A1
(43) Pub. Date: Nov. 25, 2010

(54) MicroRNA Diagnostics for Cancer

(76) Inventors: Dennis K. Watson, Mt. Pleasant, SC (US); Victoria J. Findlay, Charleston, SC (US); Omar Moussa, Mt. Pleasant, SC (US)

Correspondence Address: MUSC FOUNDATION FOR RESEARCH DEVELOPMENT, 19 HAGOOD AVE, SUITE 909, P.O. BOX 250828, CHARLESTON, SC, 29425, US

(21) Appl. No.: 12/631,201

(22) Filed: Dec. 4, 2009

Related U.S. Application Data
(60) Provisional application No. 61/119,820, filed on Dec. 4, 2008.

(51) Int. Cl. C12Q 1/68

(52) U.S. Cl. 435/6

(57) ABSTRACT

Disclosed are methods for detecting breast cancer or prostate cancer by measuring levels of miR-204 and miR-510 in samples.

### PRIORITY CLAIM

This application claims the benefit of U.S. Provisional Patent Application Ser. No. 61/119,820 filed on Dec. 4, 2008 entitled “MicroRNA Diagnostics and Therapeutics for Cancer and Uses Thereof” by Dennis K. Watson, Victoria J. Findlay and Omar Moussa, which is incorporated by reference herein as if rewritten in full.

### GOVERNMENT SUPPORT CLAUSE

This invention was made with government support under grant CA78582 awarded by the National Institutes of Health (NIH). The government has certain rights in the invention.

### BACKGROUND OF THE INVENTION

MicroRNAs (miRNAs) are noncoding RNAs that bind to target mRNAs and reduce their expression through translational repression or mRNA degradation. Measurements made in myocardial tissue have suggested the miRNAs play a regulatory role in myocardial growth, fibrosis, and remodeling. MicroRNAs have been isolated form C. elegans, Drosophila, and humans (Lagos-Quintana et al., 2001; Lau et al., 2001; Lee and Ambros, 2001). Several hundred miRNAs have been identified in plants and animals—including humans—which do not appear to have endogenous siRNAs. Thus, while similar to siRNAs, miRNAs are nonetheless distinct. miRNAs thus far observed are approximately 21-22 nucleotides in length and they arise from longer precursors, which are transcribed from non-proteinencoding genes. See review of Carrington et al. (2003). The precursors form structures that fold back on each other in self-complementary regions; they are then processed by the nuclease Dicer in animals or DCL1 in plants. miRNA molecules interrupt translation through imprecise base-pairing with their targets.

Most miRNAs are involved in gene regulation. Some of these miRNAs, including lin-4 and let-7, inhibit protein synthesis by binding to partially complementary 3′ untranslated regions (3′ UTRs) of target mRNAs. Others, including the Scarecrow miRNA found in plants, function like siRNA and bind to perfectly complementary mRNA sequences to destroy the target transcript (Grishok et al., 2001). Some miRNAs, such as lin-4, let-7, mir-14, mir-23, and bantam, have been shown to play critical roles in cell differentiation and tissue development (Ambros, 2003; Xu et al., 2003). Others are believed to have similarly important roles because of their differential spatial and temporal expression patterns.

### BRIEF DESCRIPTION OF THE INVENTION

MicroRNAs (miRNAs) are endogenous 19-25 nucleotide RNAs that have recently emerged as a novel class of small, evolutionarily conserved important gene regulatory molecules involved in many critical developmental and cellular functions 1. miRNAs base-pair with target mRNA sequences in their 3′ untranslated region (3′UTR). Through specific base pairing, miRNAs induce mRNA degradation, translational repression, or both. Each miRNA can target numerous mRNAs, often in combination with other miRNAs, therefore controlling complex regulatory networks. miRNAs have been implicated in the control of many fundamental cellular and physiological processes such as tissue development, cellular differentiation and proliferation, metabolic and signaling pathways, apoptosis and stem cell maintenance. Mounting evidence indicates that miRNAs may also play a significant role in cellular transformation and carcinogenesis acting either as oncogenes or tumor suppressors.

Disclosed are methods and materials for diagnosing the presence of breast or prostate cancer using the levels of miRNA's. The level of miRNA's can be measured in tissue samples, including samples of suspected cancerous tumors, and samples of related lymphatic tissues. The one or more miRNA's can comprise miR-204 and miR-510.

Disclosed are a subset of miRNAs (miR-28, -204, -324, -483, -486, -510, -635 and -637) that are elevated in breast and prostate cancer cell lines when compared to normal lines. We examined the expression of miR-204 and -510 in human primary breast and prostate tumor samples and found elevation of miR-204 and -510 compared to non-tumor tissue. Furthermore, we have determined that the ETS transcription factor, PDEF is a target for miR-204 and -510. PDEF is a regulator of cell migration and invasion, supporting a role of selected miRNAs in cancer progression. These miRNAs are novel oncogenes and have diagnostic and prognostic utility.

### BRIEF DESCRIPTION OF THE DRAWINGS

FIG. 1: PDEF is regulated post-transcriptionally through sequences present in the 3′UTR. (A) Quantitative real-time PCR of PDEF mRNA normalized to S26. (B) Western blot analysis of breast cancer cell lines probed with primary antibodies for PDEF and GAPDH. Semi-quantitative analysis is shown in the lower panel. (C) Schematic representation of full length, open reading frame (ORF) alone, ORF+5′UTR (5′UTR) and ORF+3′UTR (3′UTR) constructs and western blot (WB) and RT-PCR analysis of PDEF expression in MDA-MB-157 cells transiently transfected with the constructs illustrated above. (D) Luciferase activity of MDA-MB-157 cells transfected with a reporter luciferase gene (pGL3) and pGL3 fused to the PDEF 3′UTR (3′UTR) normalized to Renilla luciferase activity. The data are expressed as the mean±SD for 3 experiments conducted in triplicate.

FIG. 2: miR-204 & miR-510 regulate PDEF through its 3′UTR. (A, upper panel) Quantitative real-time PCR of miR-28 (black bars), miR-204 (white bars) & miR-510 (grey bars) normalized relative to the amount of U6 target (ΔCt). The relative levels of miRNA expression were measured by determining the ΔΔCt values of the indicated cell lines versus MCF7. (A, lower panel) Schematic representation of the predicted target sites of miR-204 & miR-510 in the 3′UTR of PDEF mRNA. The numbers (1-469) represent base pairs in the 3′UTR of PDEF and the numbers in parentheses (1425-1894) represent base pairs in the full length PDEF gene (GenBank Accession number AF071538 (42)). Complementary base sequences are highlighted in upper case letters. (B-D) Luciferase activity of HeLa cells transfected with (B) pGL3 PDEF 3′UTR reporter construct (3′UTR) and pSuppressor vector alone (pSupp), pSupp/miR-204 (miR204) or pSupp/miR-510 (miR510); (C) pGL3 reporter construct (pGL3), 3′UTR, 3′UTR reporter construct mutated in the miR204 seed sequence binding site (mut204) or 3′UTR reporter construct mutated in the miR510 seed sequence binding site (mut510); (D) or co-transfected with 3′UTR, mut204 or mut510 and pSupp (grey bars), miR204 (white bars) or miR510 (black bars). All luciferase assays were normalized to Renilla luciferase activity. The data are expressed as the mean±SD for 3 experiments conducted in triplicate.

FIG. 3: miR-204 & miR-510 regulate endogenous PDEF protein expression. (A) Western blot analysis of endogenous PDEF expression in CAMA-1 cells transfected with scrambled ASO (control) or ASO against miR-204 (204) or miR-510 (510). MCF7 and MDA-MB-231 cells are control cells positive and negative for PDEF protein expression, respectively. (B & C) Western blot analysis of MCF7 cells transfected with increasing concentrations of (B) miR-204 or (C) miR-510. Quantitative real-time PCR analysis of PDEF mRNA levels from the treatments in A, B & C normalized to GAPDH are shown to the right of the western blots.

FIG. 4: Functional consequences of miR-204 over-expression in MCF7 cells. (A) Western blot analysis of PDEF and E-Cadherin (E-Cad) expression in MCF7 control cells (MCF7) and a miR-204 over-expressing stable clone (miR204). (B) Quantitative real-time PCR of E-cadherin, uPA and SLUG mRNA expression in MCF7 control (black bars) or miR-204 over-expressing cells (grey bars) normalized to GAPDH. (C) Bright field microscopy and (D) E-Cadherin immunofluorescence of MCF7 control cells (MCF7) and a miR-204 over-expressing MCF7 stable clone (miR204).

FIG. 5: miRNA over-expression increases migration, invasion and transformed phenotypes in vitro. (A) Transwell migration (left panel) and matrigel invasion (right panel) of MCF7 control (black bars) or miR-204 over-expressing MCF7 (grey bars) cells. (B) Colony formation (left panel) and soft agar (right panel) assays of MCF7 control (black bars) or miR-204 over-expressing MCF7 (grey bars) cells. (C) Quantitative real-time PCR of uPA (left panel) and SLUG (right panel) mRNA expression of miR-204 over-expressing cells transiently transfected with PDEF (dark grey bars) or vector alone (black bars). (D) Transwell migration (left panel) and matrigel invasion (right panel) of miR-204 over-expressing cells transiently transfected with PDEF (dark grey bars) or vector alone (black bars).

FIG. 6: miR-204 & -510 levels are elevated in human breast cancer. Quantitative real-time PCR analysis of (A) miR-204 and (B) miR-510 levels in human breast tumor (grey bars) and matched non-tumor (black bars) samples (For p values see Supplementary Table 3). Representative immunohistochemical staining of PDEF in human (C) non-tumor and (D) tumor breast tissue (Case 1335; magnification ×400).

FIG. 7: PDEF protein levels do not always correlate with mRNA in breast cancer cell lines. (A) Western blot analysis of breast cancer cell lines probed with primary antibodies for PDEF and GAPDH. (B) Quantitative real-time PCR of PDEF mRNA normalized to S26.

FIG. 8: PDEF protein expression is modulated by sequences present in the 3′UTR. (A) Left panel: Schematic representation of 5′UTR+ORF, ORF alone, ORF+3′UTR and full length constructs. Right panel: Western blot analysis of PDEF expression in transiently transfected MDA-MB-157 cells probed with primary antibodies for PDEF and b-actin. (B) Luciferase activity of HEK293 cells transfected with a reporter luciferase gene (pGL3) fused to the PDEF 3′UTR.

FIG. 9: miRNAs predicted to target PDEF. (A) Quantitative real-time PCR of miR-28, -204 & -510 normalized relative to the amount of U6 target (DCt). The deregulation of miRNA expression was measured by determining the DDCt values of the indicated cell lines versus MCF7. (B) Schematic representation of the predicted target sites of miR-204 & miR-510 in the 3′UTR of PDEF mRNA. Complementary base sequences are highlighted in red.

FIG. 10: miR-204 & -510 regulate PDEF protein expression. (A) Western blot analysis of endogenous PDEF in (A) CAMA-1 cells transfected with ASO against miR-204 (204) and/or miR-510 (510) or without ASO (control) and (B & C) MCF7 cells transfected with increasing concentrations of (B) miR-204 or (C) miR-510. + and − indicate control cells positive and negative for PDEF protein expression respectively.

FIG. 11: Consequences of miR-204 overexpression. (A) Bright field microscopy showing MCF7 cells (control) and a miR-204 overexpressing stable clone (miR-204). (B) Immunofluorescence of E-cadherin in MCF7 and miR-204 cells. (C) MCF7 or miR-204 cells were seeded on a transwell insert coated with fibronectin. Cells were counted after 24 hours (*p<0.005). (D) MCF7 cells transiently transfected with empty vector or vectors containing miR-204/510 were seeded at the same density and counted after 48 hours. (E) Quantitative real-time PCR of slug, uPA and E-cadherin in MCF7 or miR-204 normalized to S26. The deregulation of mRNA expression was measured by determining the DDCt values of miR-204 versus MCF7.

FIG. 12: Proposed model for miRNA-mediated regulation of PDEF expression in breast cancer. In non-invasive breast cancer, PDEF mRNA is translated through the ribosomes resulting in PDEF protein that represses downstream targets necessary for an invasive phenotype. In invasive cancer, miRNAs bind to the 3′UTR of PDEF and inhibit translation. This leads to a loss of PDEF protein and attenuation of transcriptional repression.

FIG. 13: miR-204 & miR-510 levels correlate with high PDEF mRNA and low protein expression.

FIG. 14: miR-204 & miR-510 regulate the 3′ UTR of PDEF.

FIG. 15: miRNA inhibition increases endogenous PDEF expression.

FIG. 16: miRNA over-expression decreases endogenous PDEF expression.

FIG. 17: miR-204 over-expression leads to an EMT phenotype.

FIG. 18: Stable expression of miR-204 increases migration and invasion.

FIG. 19: Increased transformed phenotype of miR-204 over-expressing cells.

FIG. 20: miR-204 mediated modulation of downstream PDEF targets.

FIG. 21: miR-204 and miR-510 are elevated in human tumor samples.

FIG. 22: miR-510 is present in tumor samples. A human breast tissue microarray (TMA) was probed by in situ hybrization with an LNA probe against miR-510 (LNA-510; C & D) or a scrambled control (scrambled; A & B). Representative normal (A & C) and tumor (B & D) are shown. The arrow indicates a positive stain. Scale bar=50 μM.

FIG. 23: Functional consequences of miR-510 expression in normal human breast cells. Transwell migration, matrigel invasion and colony formation assays of 3 independent stable clones over-expressing miR-510 (510-1, 510-10 and 510-11) compared to vector.

FIG. 24: miR-510 is modulated by the PI3K pathway. (A) HEK293 cells were co-transfected with the pGL3 reporter vector containing a fusion of the firefly luciferase gene with the 3′UTR of PDEF (a miR-510 direct target) and Renilla luciferase vector for normalization. Cells were serum-starved and treated with various kinase inhibitors. (B & C) qPCR of Akt1 and miR-510 levels in MDA-MB-175 VII cells transduced with short hairpin Akt1 (shAkt) or scrambled control (scr).

FIG. 25: miR-510 expression activates the PI3K pathway. Western blot analysis of endogenous phospho-Akt (p-Akt), total Akt (Akt) phospho-GSK3β (p-GSK3β), and phospho-PTEN (p-PTEN) in MCF10A cells stably over-expressing miR-510 (510-1, 510-10, 510-11) or vector control (pSupp-4). GAPDH is shown as a loading control.

FIG. 26: PRDX1 expression is reduced in miR-510 expressing cells. Western blot analysis of endogenous PRDX1 levels in MCF10A cells stably over-expressing miR-510 (510-1, 510-10, 510-11) or vector control (pSupp-4). ACTIN is included as a loading control.

FIG. 27: Schematic of the method for isolating cDNA clones of mRNAs with which endogenous miR-510 forms base pairs. Cytoplasmic extract is mixed with a detergent to destabilize proteins and incubated in a reverse transcription reaction buffer to synthesize a 1st strand cDNA (black) using the miRNA (red) as a primer. Polynucleotides collected from the buffer are incubated in a 2nd strand synthesis reaction buffer to synthesize DNA (black) complementary to both the 1st strand cDNA and the miRNA. After digestion of the double stranded cDNA with a restriction enzyme, the cDNA fragments are ligated to an adapter oligonucleotide (blue) and then amplified by PCR with an adapter PCR primer (aqua) and a biotin-tagged miRNA PCR primer (aqua-biotin) corresponding to a partial sequence of the miRNA. Biotin-labeled PCR fragments are collected by binding to avidin beads and amplified by PCR with an adapter PCR primer containing an EcoRI restriction site (pink) and a nested miRNA PCR primer containing a BamHI restriction site. (pink) Amplification fragments are digested with EcoRI and BamHI, and cloned into pUC19. The sequences of inserts in randomly selected clones are determined with a vector primer.

### DETAILED DESCRIPTION OF INVENTION

PDEF is an ETS transcription factor expressed in normal tissues with high epithelial cell content and non-invasive breast cancer cells. A putative tumor suppressor, PDEF protein expression is often lost during progression to a more invasive phenotype. Interestingly, PDEF mRNA has been found to retained or even over-expressed in the absence of protein; however, the mechanisms for this remain to be elucidated. Disclosed are two microRNAs that directly act on and repress PDEF mRNA translation, leading to the loss of PDEF protein expression and the gain of phenotypes associated with invasive cells. In addition, we show that these microRNAs are elevated in human breast tumor samples. Together, these data describe a mechanism of regulation that explains for the lack of correlation between PDEF mRNA and protein levels, providing insight into the under-explored role of post-transcriptional regulation and how this contributes to dysregulated protein expression in cancer.

Cancer death is due in large part to metastases. One of the more interesting challenges is to understand the cellular changes that occur during progression towards invasive cancer. ETS proteins are a large family of transcription factors with diverse functions and activities that activate or repress the expression of genes that are involved in various biological processes, including cellular proliferation, apoptosis, differentiation, and transformation (1, 2). The ETS family gene, PDEF (prostate derived epithelial factor), is expressed in normal epithelial tissues including prostate, breast, and colon (3). In normal tissue and non-invasive cancers, mRNA and PDEF protein are easily detectable by northern and western blot. However, PDEF protein loss is correlated with prostate, breast, and colon cancer progression to an invasive phenotype both in vitro and in vivo (3-5). Interestingly, this loss of protein does not always correlate well with PDEF mRNA levels (5). Indeed, some invasive cancers retain or have elevated levels of PDEF mRNA in the absence of protein (3, 6, 7). PDEF re-expression in multiple invasive prostate, breast, and colon cancer cells results in reduced cell growth, migration and invasion (2, 3) (and unpublished data). Reciprocal siRNA-mediated knockdown experiments in PDEF expressing non-invasive cells results in increased migration and invasion together with an altered morphology consistent with a more invasive phenotype (2). Together, these and other data support the model that PDEF target genes control several aspects of the multi-step metastatic process and specifically, loss of PDEF regulatory networks is a key event in the development of invasive cancer (8).

MicroRNAs (miRNAs) are endogenous 19-25 nucleotide non-coding RNAs that have recently emerged as a novel class of small, evolutionarily conserved important gene regulatory molecules involved in many critical developmental and cellular functions (9). Through specific base pairing with target mRNA sequences in the 3′ untranslated region (3′UTR), miRNAs induce mRNA degradation, translational repression, or both (10). Individual miRNAs can target numerous mRNAs, often in combination with other miRNAs, thereby providing a mechanism for controlling complex regulatory networks. It is estimated that there are over 600 miRNAs in mammalian cells, and that about 30% of all genes are regulated by miRNAs (11, 12). Over 3,000 identified mature miRNAs exist in species ranging from plants to humans. Their existence and conservation throughout species supports the concept that they perform critical functions in gene regulation (13). Indeed, the conserved evolution of both miRNAs and transcription factors highlights their importance in and the complexity of gene regulation (14). miRNAs have been implicated in the control of many fundamental cellular and physiological processes, including tissue development, cellular differentiation and proliferation, metabolic and signaling pathways, apoptosis and stem cell maintenance (15-17). Mounting evidence indicates that miRNAs may also play a significant role in cellular transformation and carcinogenesis acting either as oncogenes or tumor suppressors (18, 19). Hence, there are few cellular processes that are not affected by miRNAs. In addition, specific miRNA signatures have been identified for both solid cancers and hematologic malignancies (20-23), and mounting evidence suggest that the power of miRNAs lies in the ability to distinguish specific cancer subtypes based on their miRNA profile, including, and of direct relevance to the studies described herein, breast cancer (20, 24). Nonetheless, the identification and validation of specific targets has been limited. Disclosed here is PDEF as a novel target for miR-204 and miR-510 and a mechanism for the loss of PDEF protein expression during breast cancer progression.

### Experimental Procedures

Cell Culture. Human breast cancer cell lines (MCF7, BT474, CAMA-1, HBL100, HCC202, Hs578t, MDA-MB-157, MDA-MB-175 VII, MDA-MB-231, MDA-MB-361, MDA-MB-415, MDA-MB-436 and MDA-MB-453) were cultured according to the ATCC website. The breast cancer cell lines CAMA-1, HBL100, HCC202, MDA-MB-415 and MDA-MB-436 were a kind gift of R. Neve (University of California, CA). All other lines were obtained from ATCC. For the generation of stable MCF7 cells overexpressing miR-204, pSuppressor-neo vector (Imgenex; San Diego, Calif.) expressing miR-204 was transfected into MCF7 cells and stable cells were selected in medium containing G418 (Invitrogen; Carlsbad, Calif.). This vector system was also used for transient expression of miR-204 and miR-510 into MCF7 cells.

Tumor samples. Matched tumor and non-tumor breast samples were obtained from the Hollings Cancer Center tumor bank at MUSC. Prior to surgery at the Center, all patients provided written informed consent to allow any excess tissue to be used for research studies. Samples were snap frozen in OCT and stored at −80° C. until use. The pathological status of the specimens was confirmed by histological examination of 10 uM sections taken at the start, middle and end of the 5×20 μM sections taken for RNA analysis. Each tumor section contained between 65-80% malignant epithelial cells and 0-5% non-malignant epithelial cells. Non-tumor sections contained 100% benign epithelial cells. To determine PDEF expression, human breast cancer paraffin blocks of tissues available from the same patients were obtained from the HCC Tumor Bank (MUSC).

Immunohistochemistry. Antigen retrieval was done by heating in a microwave oven for 2×5 min on half power in 10 mmol/L citrate (pH 6.0). Sections were washed, treated with 1% H₂O₂ for 15 min and non-specific binding was blocked with 2.5% horse serum (ImmPRESS Vector staining kit; Vector Laboratories, Burlington, Calif.) for 1 h and then incubated overnight at 4° C. with PDEF primary antibody at a 1:100 dilution in 2% BSA in PBS. Overnight incubation at 4° C. was followed by 3×10 min washes in PBS, Immpress anti-rabbit secondary antibody was incubated (Vector Laboratories) for 2 h at room temperature. After washing with H₂O, 3,3′-diaminobenzidine substrate (Sigma, St Louis, Mo.) was added for 2 min followed by washing in H₂O, Slides were counterstained with hematoxylin.

Quantitative reverse transcription PCR. Total RNA from breast tissues and cancer cell lines was extracted using the RNeasy Plus Mini Kit (Qiagen; Valencia, Calif.). One microgram total RNA was reverse transcribed in a 20 μl reaction using Superscript III reverse transcriptase (Invitrogen) for microRNA analyses and iScript (Bio-Rad; Hercules, Calif.) for all other studies. Real time PCR was performed with 1 μl of a 1:10 dilution of reverse transcribed cDNA using the Platinum SYBR Green qPCR SuperMix UDG (Invitrogen) in a LightCycler (Roche, Nutley, N.J.). The cycling conditions for all genes were: pre-incubation at 50° C. for 2 minutes, 95° C. for 2 minutes, followed by 30-50 cycles of denaturation at 94° C. for 10 seconds, annealing at one degree below the lowest Tm for each gene-specific primer pair (Supplemental Table 1) for 10 seconds and extension for 30 seconds at 72° C., with a single data acquisition at the end of each extension. All ramping was done at 20° C. per second. Triplicate reactions were run for each cDNA sample. The relative expression of each gene was quantified on the basis of Ct value measured against an internal standard curve for each specific set of primers (Supplemental Table 1) using the software provided by the instrument manufacturer (Roche). These data were normalized to S26, GAPDH or U6 (see individual experiments). The size and purity of the PCR products were also analyzed by agarose gel electrophoresis and visualized by ethidium bromide staining.

Plasmid construction. To over-express miR-204 and miR-510, the genomic region surrounding the pri-microRNA sequence of miR-204 and miR-510 were amplified with ThermalAce (Invitrogen). The cycling conditions were: pre-incubation at 94° C. for 5 minutes, followed by 30 cycles of denaturation at 94° C. for 1 minute, annealing at 65° C. for 1 minute and extension for 1 minute at 72° C. The PCR products (˜500 bp) were directionally cloned into the pSuppressor-neo vector (Imgenex) using XhoI and XbaI. The 5′UTR, ORF, 3′UTR and full length sequences of PDEF were amplified from human genomic DNA of MCF7 cells and directionally cloned into pcDNA3 (Invitrogen). The wild type 3′UTR of PDEF was cloned into the XbaI site of the pGL3-promoter vector (Promega; Madison, Wis.). The sequences complementary to the seed of the miR-204 and miR-510 were deleted using a QuikChange Site-Directed Mutagenesis Kit (Stratagene; La Jolla, Calif.). For primer sequences and Ta's, see Supplementary Table 2. All constructs were validated by sequencing at the MUSC sequencing facility.

Oligonucleotide Transfection. The miRNA inhibitors (Ambion; Austin, Tex.) are single-stranded chemically enhanced oligoribonucleotides designed to inhibit the endogenous miRNAs. Cells were transfected with 100 nM of the indicated oligoribonucleotide using the Oligofectamine reagent as per the manufacturer's instructions (Invitrogen). 48 hours after transfection, cells were harvested for protein or RNA extraction.

Luciferase assays. Cells were plated at 200,000 cells per well in a 6-well plate. The pGL3 reporter constructs (0.5 μg, firefly luciferase) were co-transfected with pRL-TK (0.05 μg, Renilla luciferase) using Lipofectamine 2000 as per the manufacturer's instructions (Invitrogen). The media was changed the next day, and luciferase activity measured after 48 h using the dual luciferase reporter assay system (Promega). Firefly luciferase activity was normalized to Renilla luciferase activity for each transfected well.

Western blot analysis. Cell lysate preparation and western blot analysis using enhanced chemiluminescence were performed as described previously (2). Experimental antibodies include human PDEF (prepared as described previously (3)) and E-Cadherin (BD BioSciences, San Jose, Calif.). GAPDH and beta-actin (Abcam, Cambridge, Mass.) were used as loading controls.

Transwell migration and invasion assay. Stably transfected MCF7 cells were seeded into the upper chamber of a Transwell insert pre-coated with 5 μg/ml fibronectin for migration or a BD™ Matrigel invasion chamber for invasion, in serum-free medium at a density of 50,000 cells per well (24-well insert; pore size, 8 μM; BD Biosciences). Medium containing 10% serum was placed in the lower chamber to act as a chemoattractant, and cells were further incubated for 24 h. Non-migratory cells were removed from the upper chamber by scraping with a cotton bud. The cells remaining on the lower surface of the insert were stained using Diff-Quick (Dade Behring, Inc., Newark, Del.). Cells were quantified as the number of cells found in 10 random microscope fields in two independent inserts. Error bars represent the SD from three separate experiments.

Immunofluorescence. Cells were seeded onto sterile cover slides (18 mm diameter) coated with 5 μg/ml fibronectin and allowed to attach overnight. Cells were then fixed with 2% formaldehyde, permeabilized with 0.1% Triton X-100 and blocked in 2% BSA for 1 h at room temperature. E-Cadherin expression was examined using the antibody detailed above and visualized using Alexa Fluor secondary antibody (Invitrogen). Immunofluorescence was examined using an Olympus IX70 confocal microscope.

Colony formation Assay. Wild type and miR-204 stably transformed MCF7 cells were seeded at a cell density of ˜4 cells/mm² in normal growth media. Cells were incubated as normal and colonies were counted after 7-10 days.

Soft-agar assay. 2 ml of 0.6% agarose in 2×DMEM was plated in each well of a 6-well plate and left to set for 20 min. This layer was overlaid with 1.0×10⁴ wild type and miR-204 stably transfected MCF7 cells in 3 ml of 0.4% agarose diluted in 2×DMEM. Cells were incubated as normal for ˜14 days and the colonies counted.

Statistical analysis. For statistical testing, two-sided paired Student's t-tests were done using Excel spreadsheet. p values are given for each individual experiment, but in general, p<0.05 was considered statistically significant. Error bars represent standard deviations of three independent experiments unless indicated otherwise.

### Results

PDEF is post-transcriptionally regulated during breast cancer progression

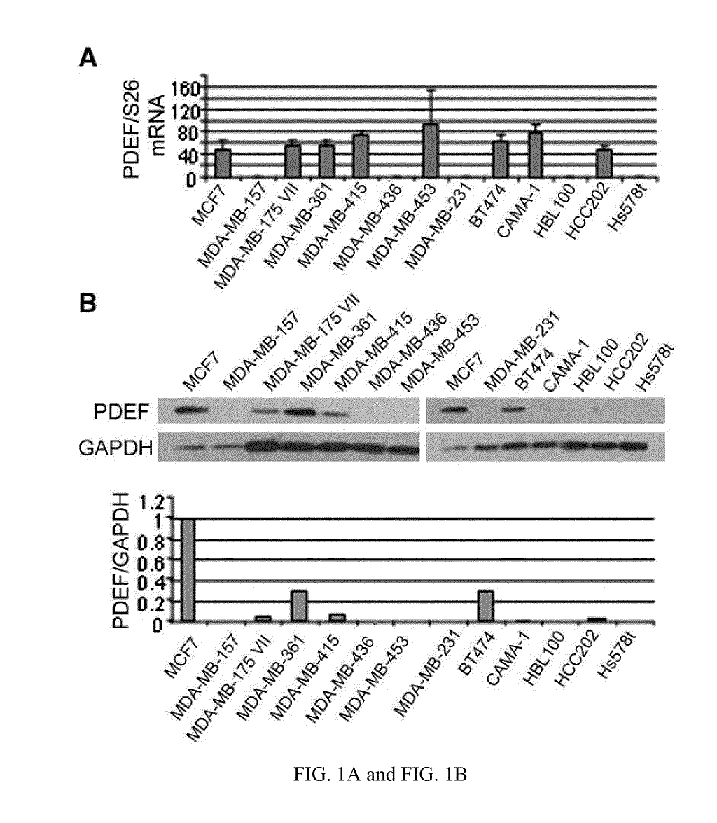

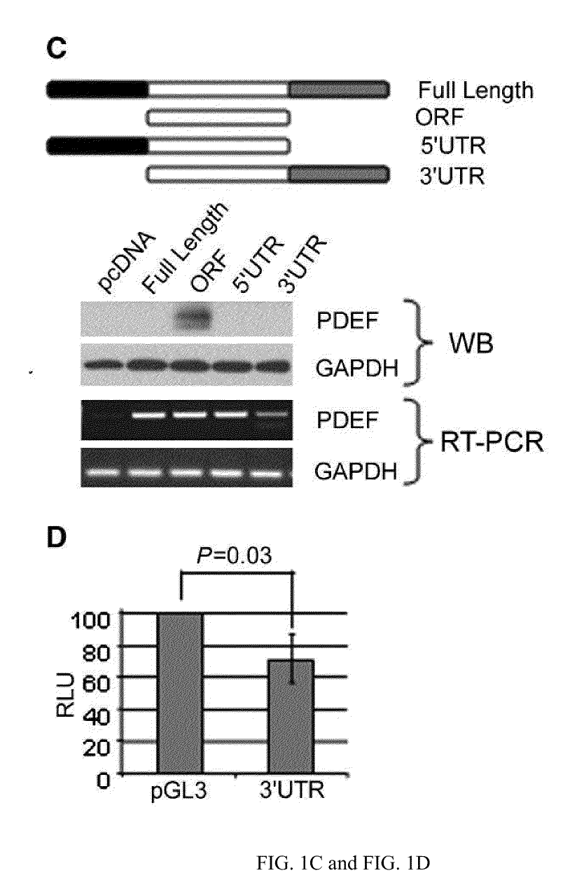

The discordance between PDEF mRNA and protein levels supported the model that PDEF may be post-transcriptionally regulated during breast cancer progression. To identify appropriate cell line systems to examine this model, we evaluated PDEF mRNA and protein levels in multiple breast cancer cell lines by real-time PCR and western analyses, respectively. MCF7, MDA-MB-361 and BT474 cell lines expressed detectable levels of both PDEF mRNA and protein, and MDA-MB-175 VII, MDA-MB-415, MDA-MB-453, CAMA-1 and HCC202 cell lines had PDEF mRNA, but little or no detectable levels of protein (FIGS. 1A & B). These cell lines are derived from luminal cells and appear more differentiated, form tight cell-cell junctions and are non- or weakly invasive (25). In contrast, MDA-MB-157, MDA-MB-436, MDA-MB-231, HBL100 and Hs578t cells had neither PDEF mRNA or protein (FIGS. 1A & B) and are derived from basal B cells, appear less differentiated, have a more mesenchymal-like appearance and are highly invasive (25). Therefore, cells lose PDEF protein during progression to a more invasive cancer.

Our previous gain-of-function studies utilized a construct expressing only the open reading frame (ORF) of PDEF to over-express the protein in multiple breast cancer cell lines (2, 3). These observations supported the notion that PDEF may be regulated post-transcriptionally through sequences in its untranslated regions (UTR). To study UTR-dependent translational control of PDEF transcripts, the level of PDEF mRNA and protein expression following transfection of MDA-MB-157 cells with constructs lacking 5′ and/or 3′ UTR sequences were compared with that allowing expression of the full-length PDEF transcript (FIG. 1C). RT-PCR analysis demonstrated that PDEF mRNA is expressed following transfection of each construct (FIG. 1C). In contrast, PDEF protein was not detectable in cells transfected with PDEF constructs containing both or either UTR (FIG. 1C), supporting the model that elements present within the 3′ UTR of PDEF negatively regulate its mRNA translation in breast cancer. One mechanism of post-transcriptional regulation is through miRNA-mediated repression of elements present in the 3′UTR of genes. To investigate whether PDEF mRNA translation was regulated by factors binding to its 3′UTR, we fused the 3′UTR of PDEF mRNA to a luciferase reporter. The presence of the 3′UTR of PDEF resulted in a significant reduction in luciferase activity compared with the unmodified control luciferase reporter (FIG. 1D).

MicroRNAs Regulate PDEF Expression Through its 3′UTR

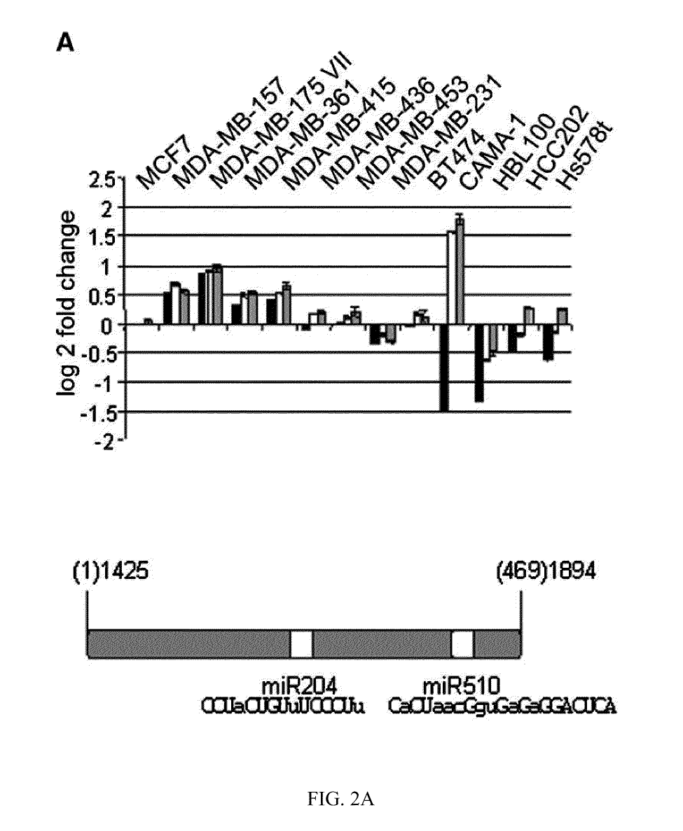

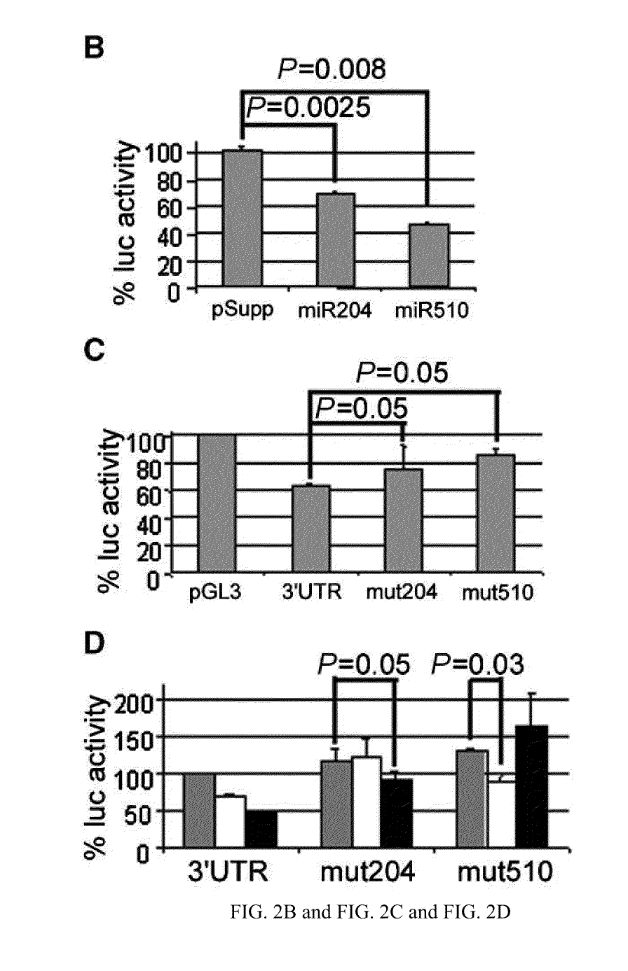

To determine whether miRNAs played a role in the post-transcriptional regulation of PDEF expression, we conducted a bioinformatics analyses and identified twelve potential miRNA recognition sequences within the 3′ UTR of the PDEF transcript. Primers were designed for the top ten scoring miRNAs. Real-time PCR analysis on a series of breast cancer cell lines revealed that, compared to 8 other miRNAs, the expression of two miRNAs, miRNA 204 (miR-204) and miRNA 510 (miR-510), were most correlated with breast cancer cell lines having high PDEF RNA and low PDEF protein levels (FIG. 2A and data not shown). Specifically, miR-204 and miR-510 are expressed at elevated levels relative to MCF7 in ⅘ and 5/5 of the breast cancer cell lines with high PDEF RNA and low PDEF protein (MDA-MB-175 VII, MDA-MB-415, MDA-MB-453, CAMA-1 and HCC202), while miR-28 is elevated in only ⅖ lines (MDA-MB-175 VII, MDA-MB-415). Although some elevated expression of miR-204 & -510 was also observed in other breast cancer cell lines when compared to MCF7, we chose these two miRNAs as the best candidates of the ten initially characterized for further investigation in the negative regulation of PDEF expression. The predicted sites for miRNAs within a 3′UTR can overlap or even be identical; however, miR-204 and miR-510 bind to separate sites within the 3′UTR of PDEF (FIG. 2A). To examine whether PDEF expression is repressed by miR-204 and/or miR-510 through these elements, the luciferase reporter construct containing the 3′UTR of PDEF was co-transfected into HeLa cells together with a plasmid construct designed to over-express either miR-204 or miR-510. Co-transfection with either miR-204 or miR-510 further reduced the luciferase activity, to 55% and 35%, respectively (FIG. 2B). The most important criteria for target recognition are the 5′ five to eight nucleotide core sequence of a miRNA, known as the ‘seed sequence’. To further validate that miR-204 and miR-510 are direct repressors of PDEF via binding to the identified sites within the 3′UTR, we mutated the seed sequences of miR-204 and miR-510, respectively, in the luciferase reporter construct. Transfection of HeLa cells with these mutated luciferase reporters resulted in ˜25% and 40% increase in luciferase activity, respectively (FIG. 2C). Furthermore, miR-204 and miR-510 had no significant repressive effect on luciferase activity when over-expressed in cells transfected with luciferase-UTR constructs containing the respective mutated seed sequences (FIG. 2D).

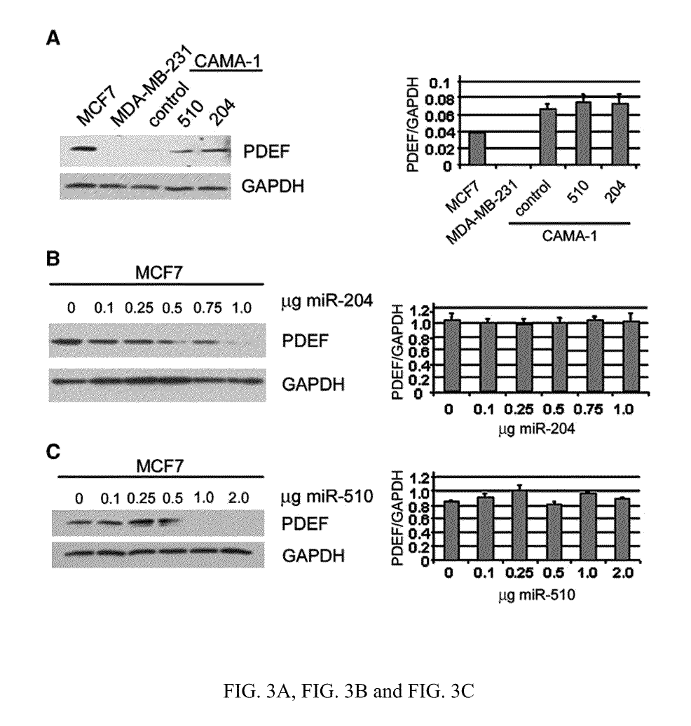

Regulation by miRNAs of protein translation can be due to translational repression and/or mRNA degradation. To assess whether miR-204 and/or miR-510 have a functional role in the down-regulation of endogenous PDEF expression by either of these mechanisms, two cell lines were selected as model systems: CAMA-1 (elevated PDEF mRNA, low PDEF protein) and MCF7 (PDEF mRNA and protein). Transfection of antisense oligoribonucleotides (ASO) targeted against miR-204 or miR-510 in CAMA-1 cells resulted in an increase in PDEF protein levels, while the PDEF mRNA levels remained unchanged (FIG. 3A). Reciprocal over-expression of either miR-204 or miR-510 in MCF7 cells results in a loss of PDEF protein, without significantly affecting the levels of PDEF mRNA (FIGS. 3B & C). PDEF protein is present at very low (miR-204) or undetectable levels (miR-510) in cells transfected with 1 μg plasmid. These results demonstrate that miR-204 and miR-510 post-translationally regulate endogenous PDEF mRNA, most likely through a mechanism of translation inhibition.

Functional Consequences of the Regulation of PDEF by miR-204

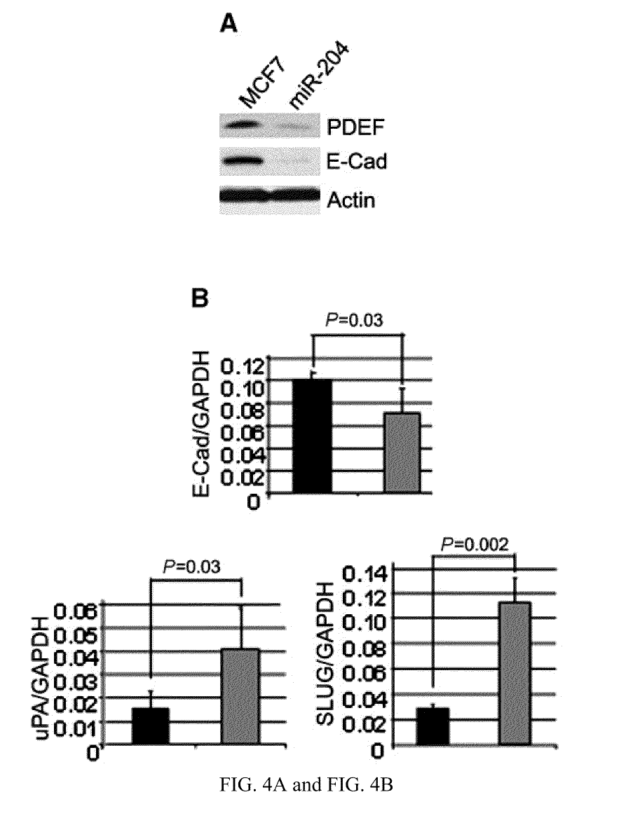

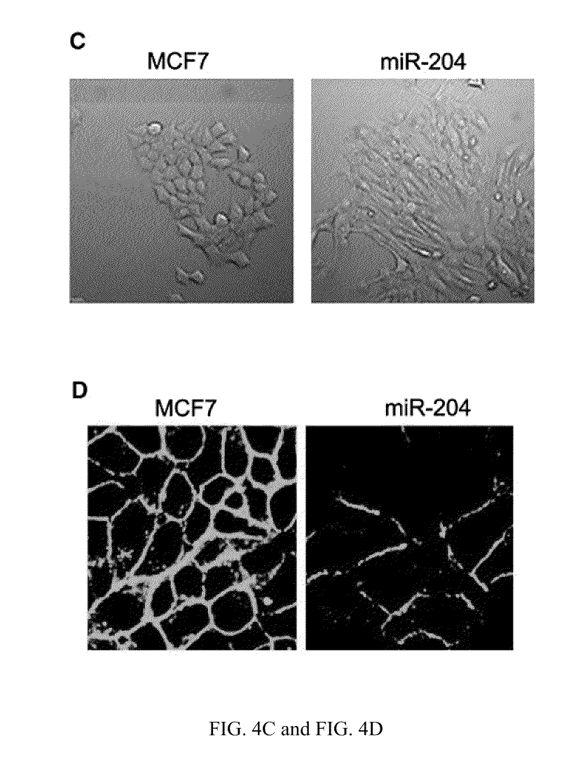

Studies in our laboratory have shown that the over-expression of PDEF in an invasive breast cancer cell line leads to an altered cell morphology (2, 3). MCF7 cells typically have an epithelial morphology described as cobblestone in appearance. However, MCF7 cells that were stably transfected with miR-204 and showed concomitant lower levels of PDEF protein expression (FIG. 4A) had an altered cell morphology (FIG. 4C). These cells also had a more spindle-like morphology and appeared more mesenchymal, a change similar to that observed in cells that have undergone an EMT (epithelial-to-mesenchymal transition) (26). PDEF is a transcription factor involved in the negative regulation of genes involved in metastatic progression. Studies in our laboratory have identified uPA and slug as negative PDEF response genes, as their mRNA expression levels decrease when PDEF is over-expressed (3) (and unpublished data), and as direct targets as shown by chromatin immunoprecipitation (Turner et al, manuscript in preparation). uPA and slug are known to be involved in metastatic progression and slug up-regulation plays distinct roles during EMT (27, 28) and therefore these genes were selected for examination in our miR-204 stable over-expressing cells. Consistent with negative regulation by PDEF, miRNA-mediated reduction in PDEF protein expression resulted in an increase in the levels of both uPA and slug by quantitative real-time PCR analysis (FIG. 4B). The adhesion protein E-cadherin plays critical roles during epithelial morphogenesis (29). Expression of this protein is down-regulated during the acquisition of invasive and metastatic phenotypes at late stages of epithelial tumor progression. Slug is established as a transcriptional repressor of E-cadherin gene expression in this process (28). Therefore, we performed quantitative real-time PCR analysis and confirmed that E-Cadherin mRNA levels were reduced in miR-204 over-expressing cells (FIG. 4B). Concurrently, we observed a decrease in total and surface staining of E-Cadherin by immunofluorescence (FIG. 4D) and total protein levels by western blot analysis (FIG. 4A).

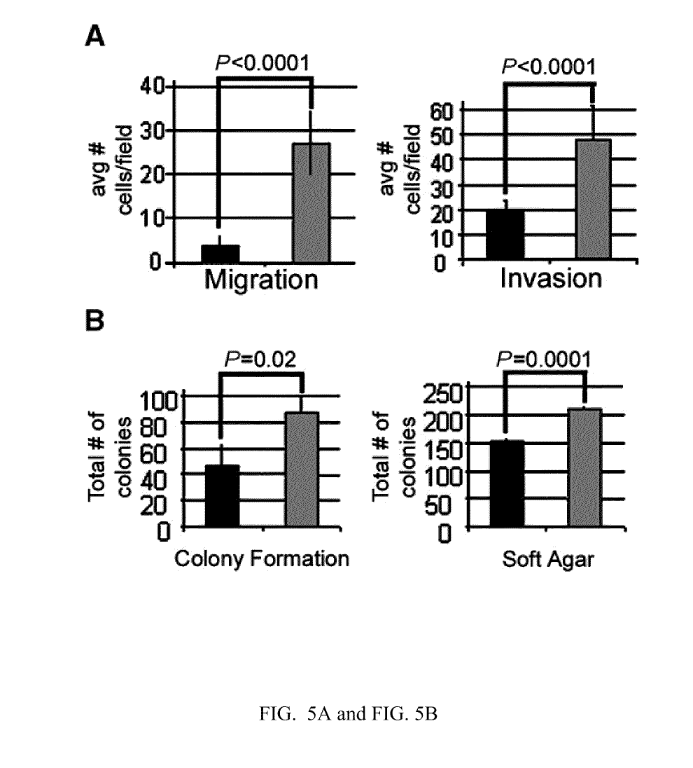

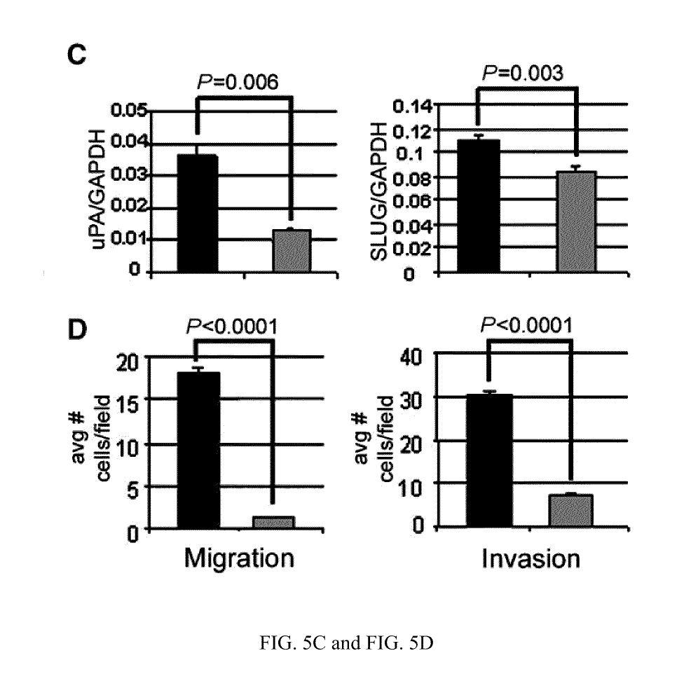

Cells that have undergone an EMT have higher migratory and invasive properties. To assess whether migration and/or invasion are altered in miR-204 over-expressing cells, we performed transwell migration assays across a chemokine gradient and invasion assays through matrigel. The number of cells found to migrate or invade in miR-204 over-expressing cells was significantly increased compared to the parental control (FIGS. 5A & B). To further explore the possible role of miR-204 in cancer progression, we investigated the ability of the stably transfected MCF7 cells to proliferate when seeded at low density in vitro using a clonogenic assay and observed an increase in the total number of colonies formed when compared to the parental control (FIG. 5C). A hallmark of transformed cells is their ability to grow independent of anchorage; therefore, to determine whether the over-expression of miR-204 resulted in enhanced transformation, we performed a soft agar assay and observed an increase in the total number of colonies that were able to form in the cells over-expressing miR-204 (FIG. 5D). Each of these molecular and cellular phenotypes were confirmed with multiple miR-204 stable clones (data not shown). Similar findings were obtained for cells following transient expression of miR-510 in MCF7 cells (Supplementary FIG. 1).

PDEF Expression Inhibits the miR-204 Over-Expressing Phenotype

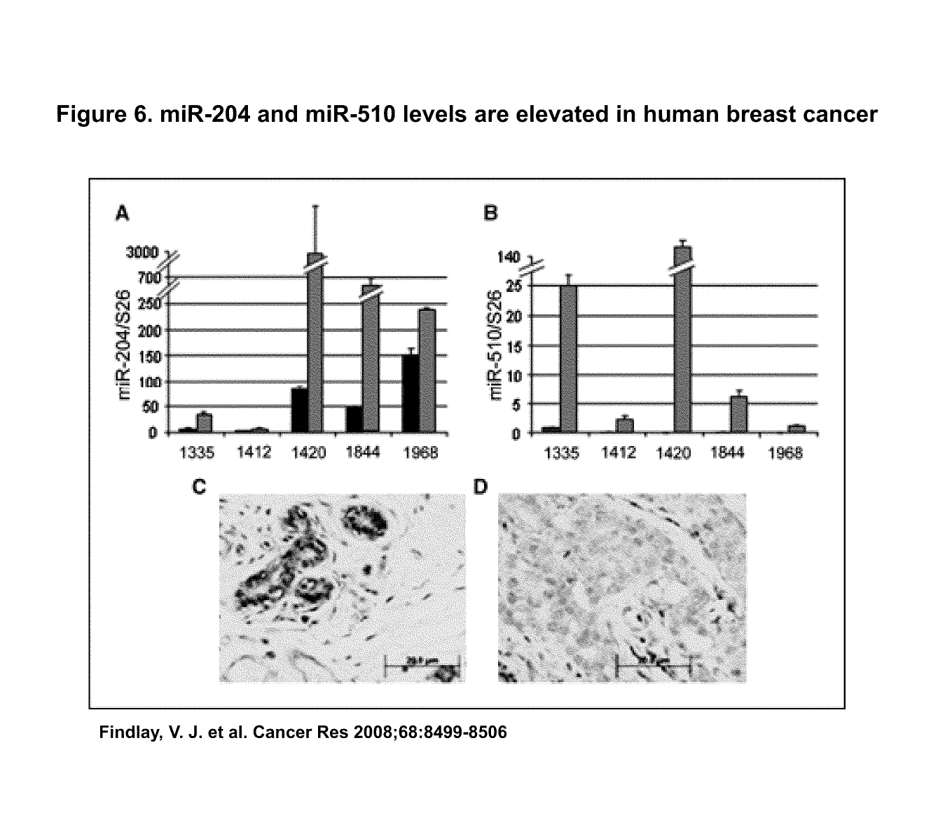

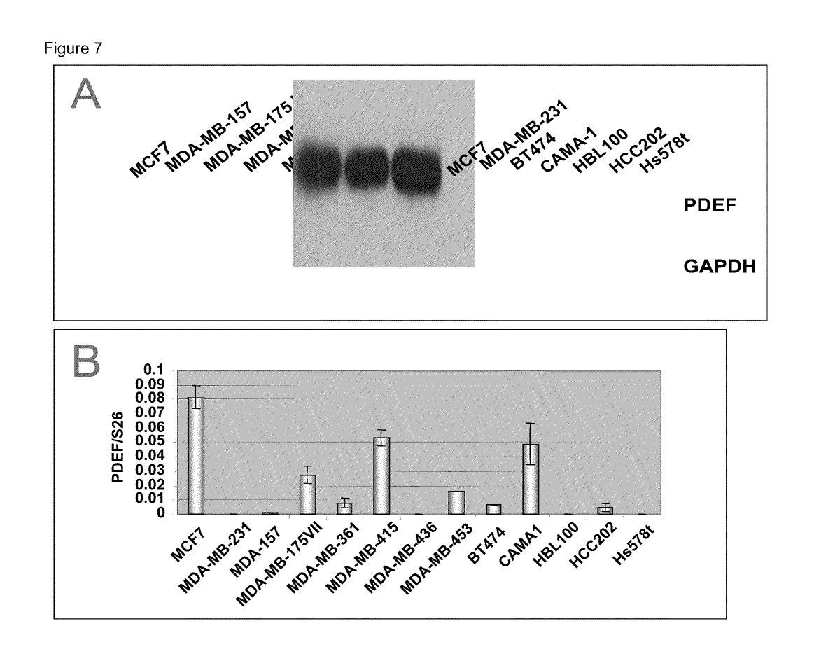

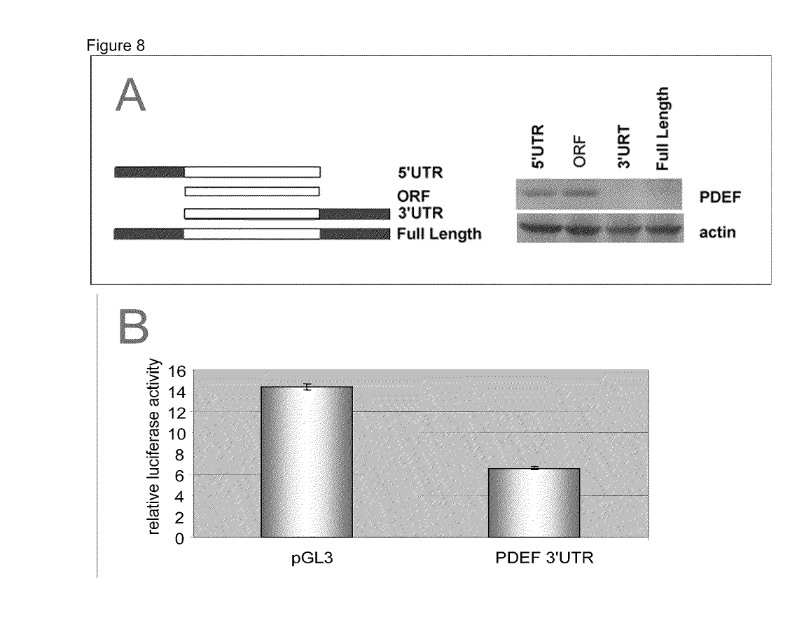

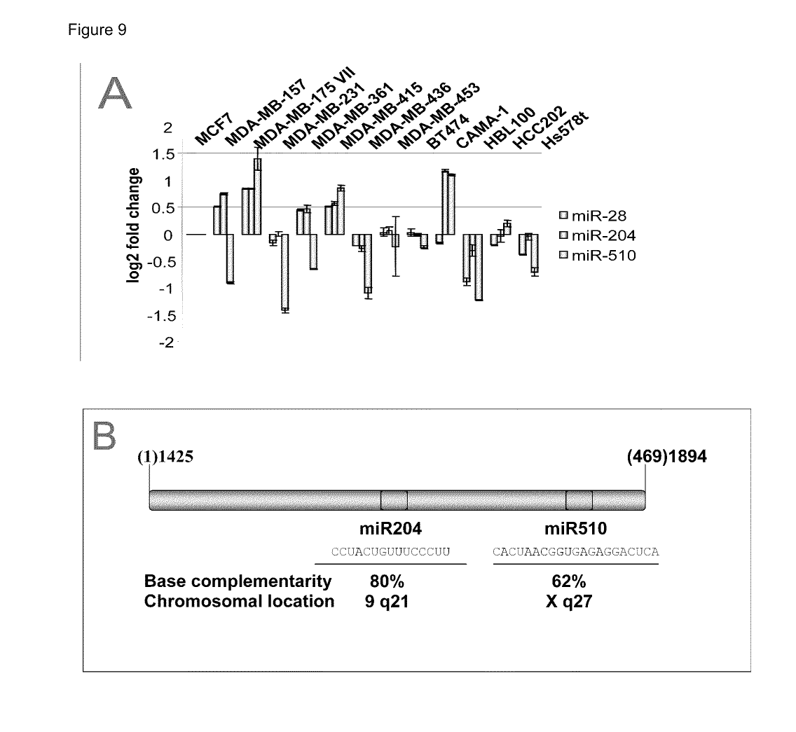

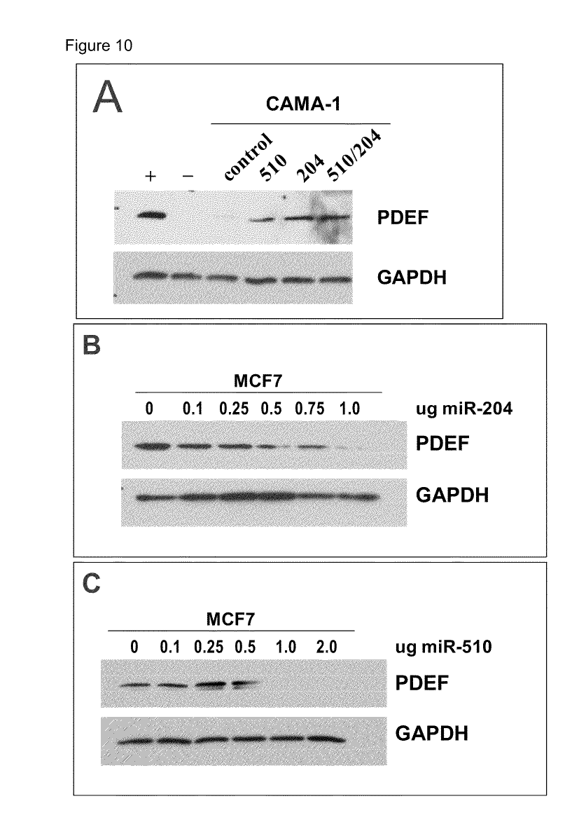

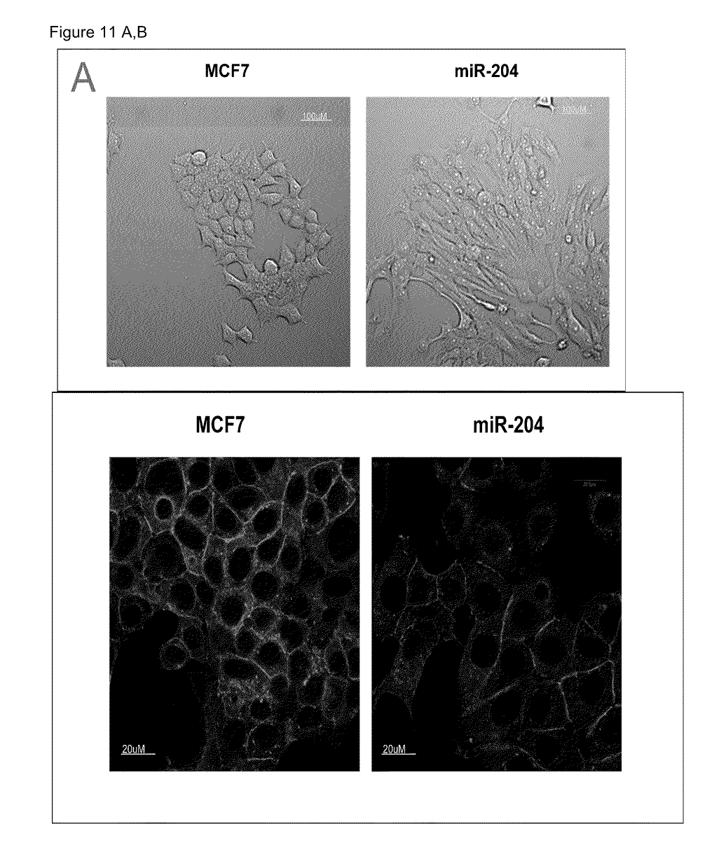

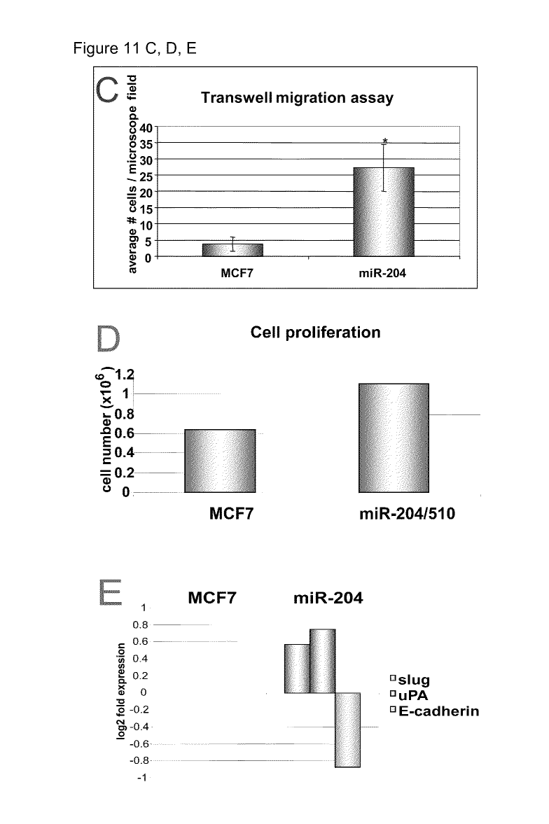

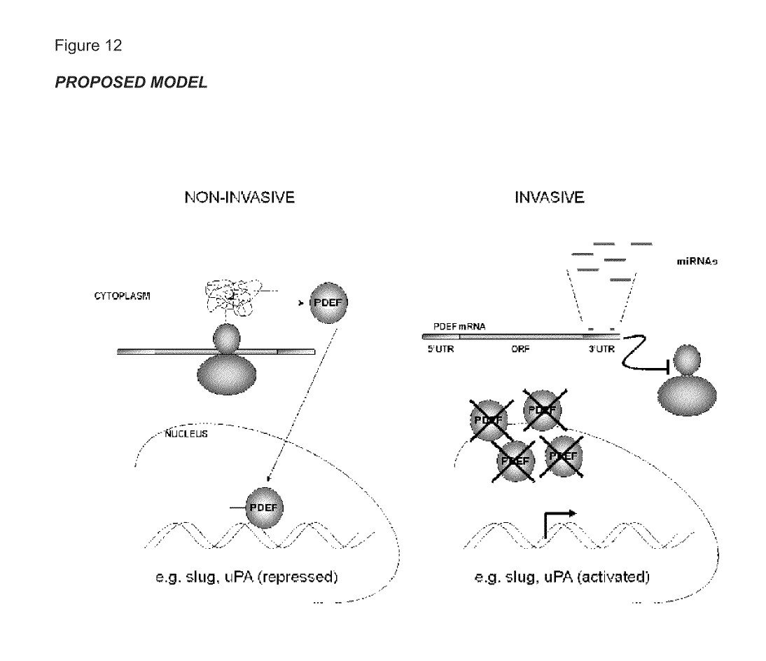

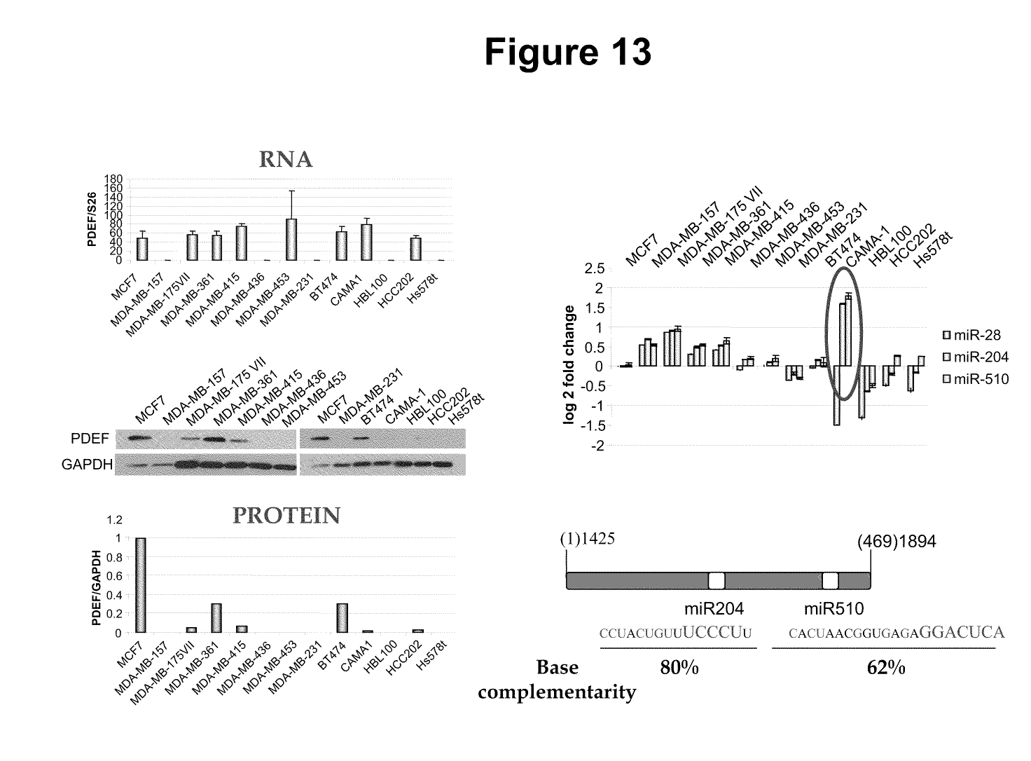

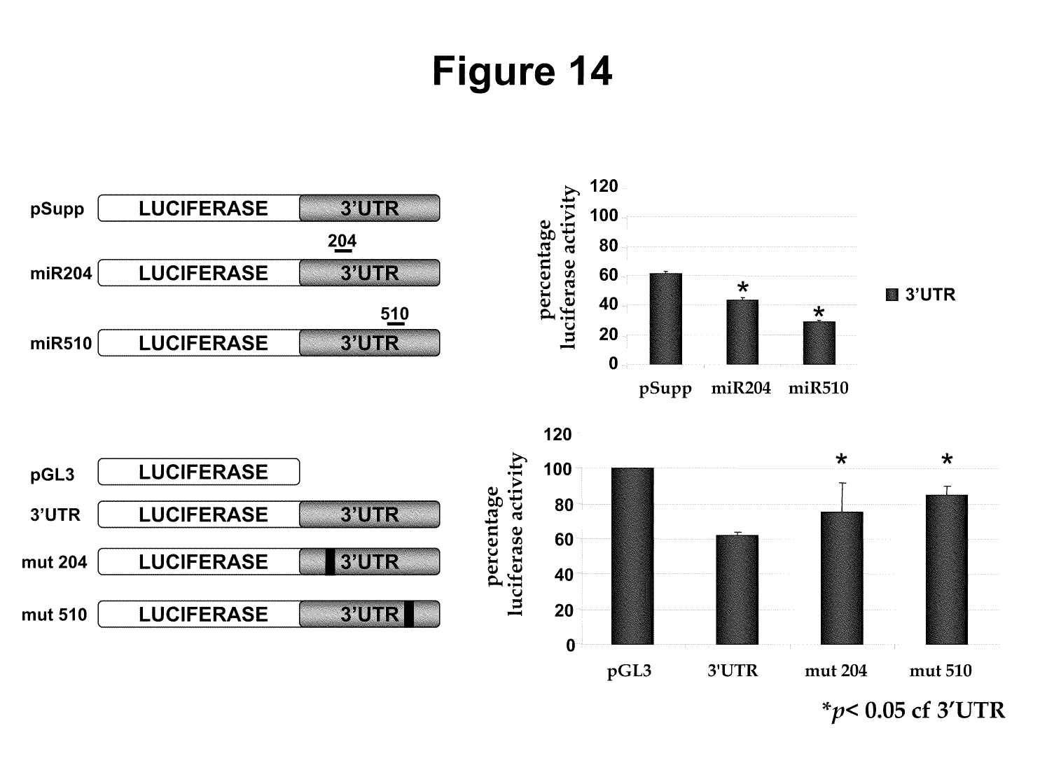

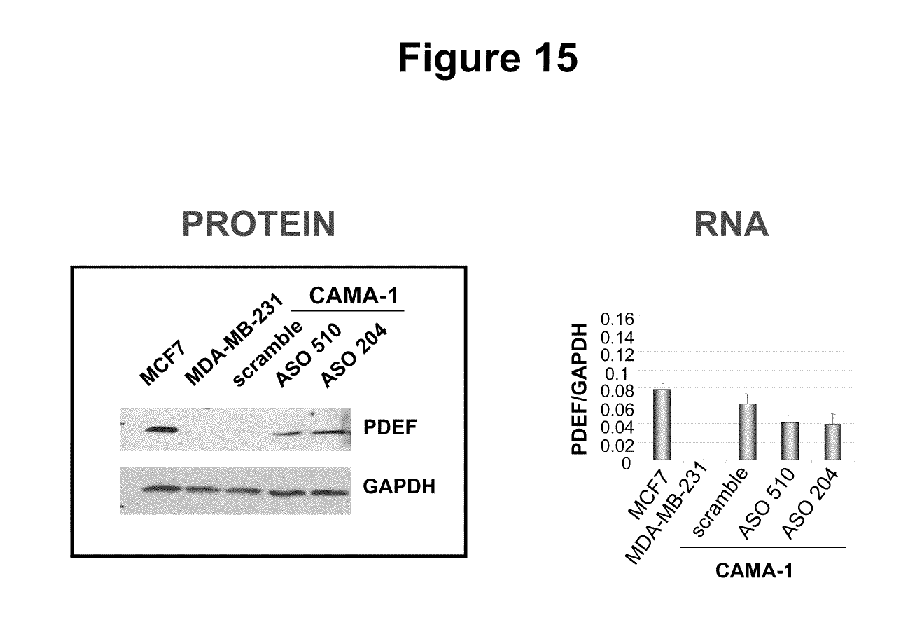

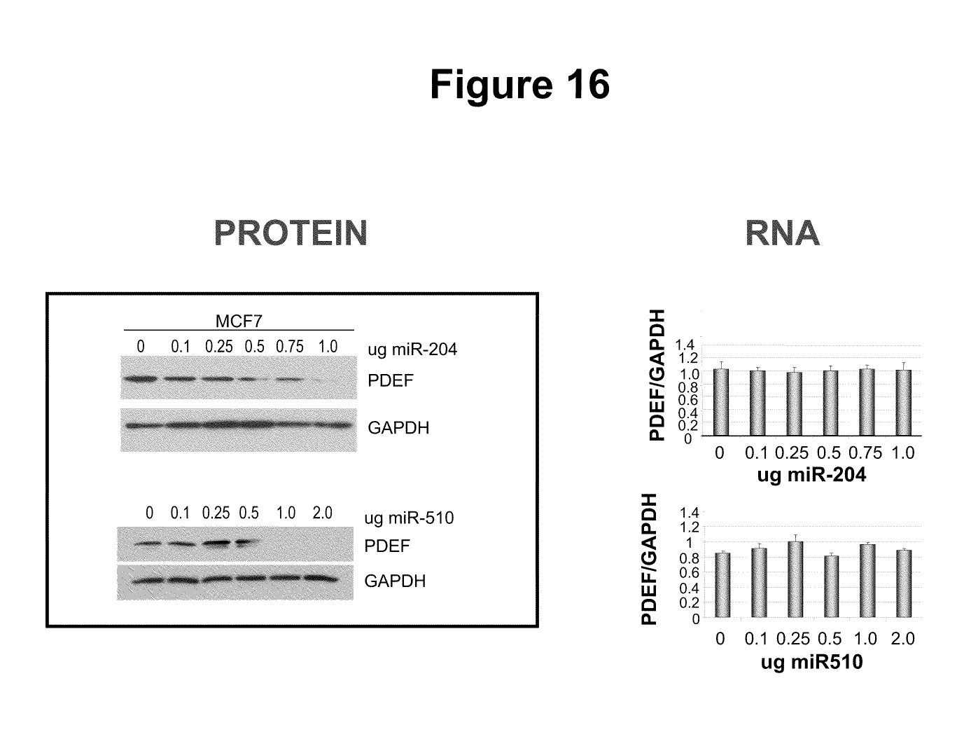

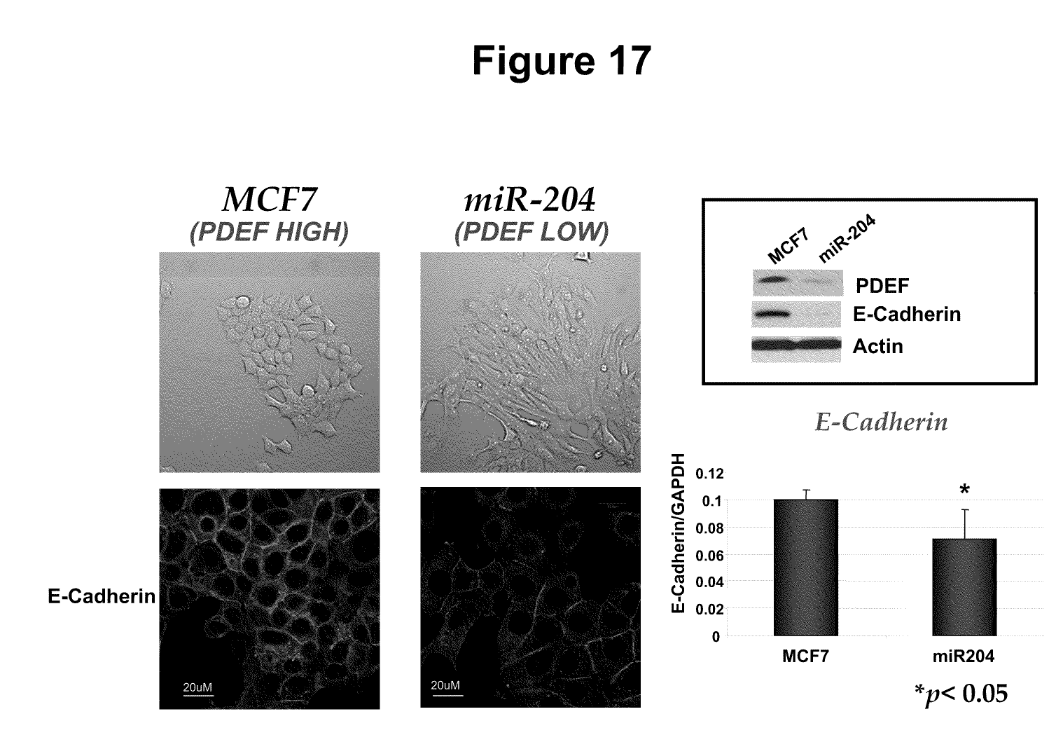

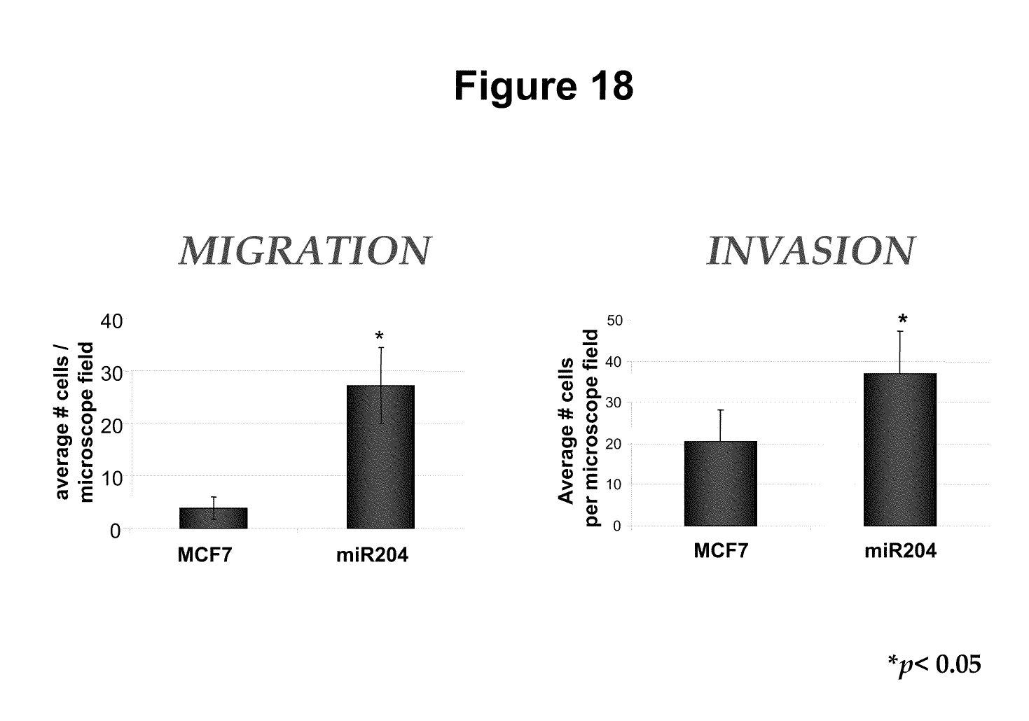

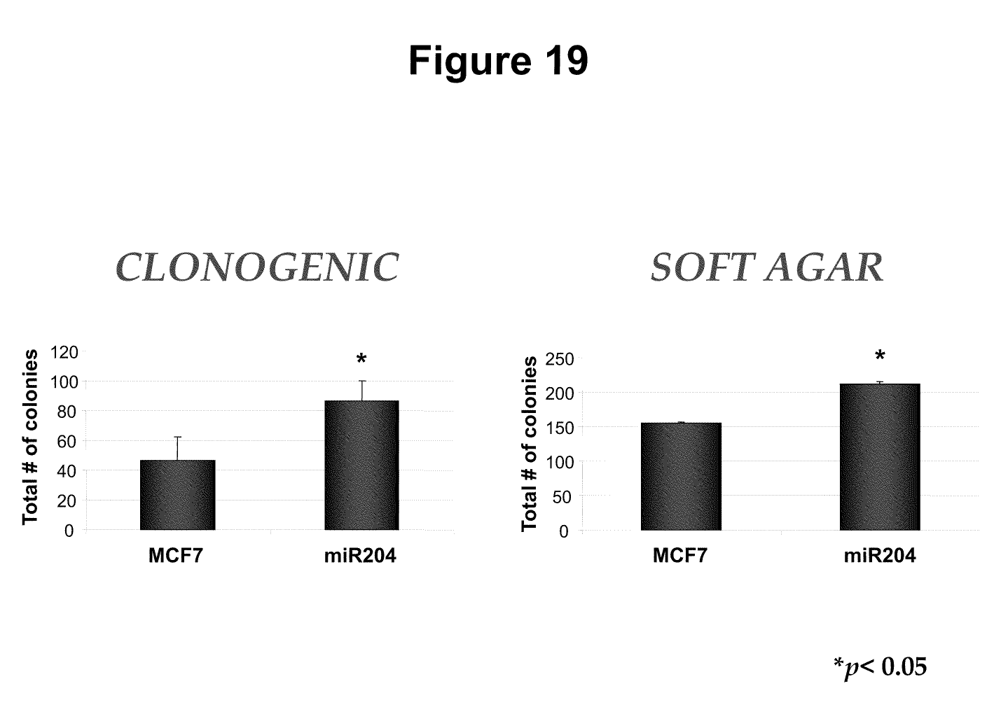

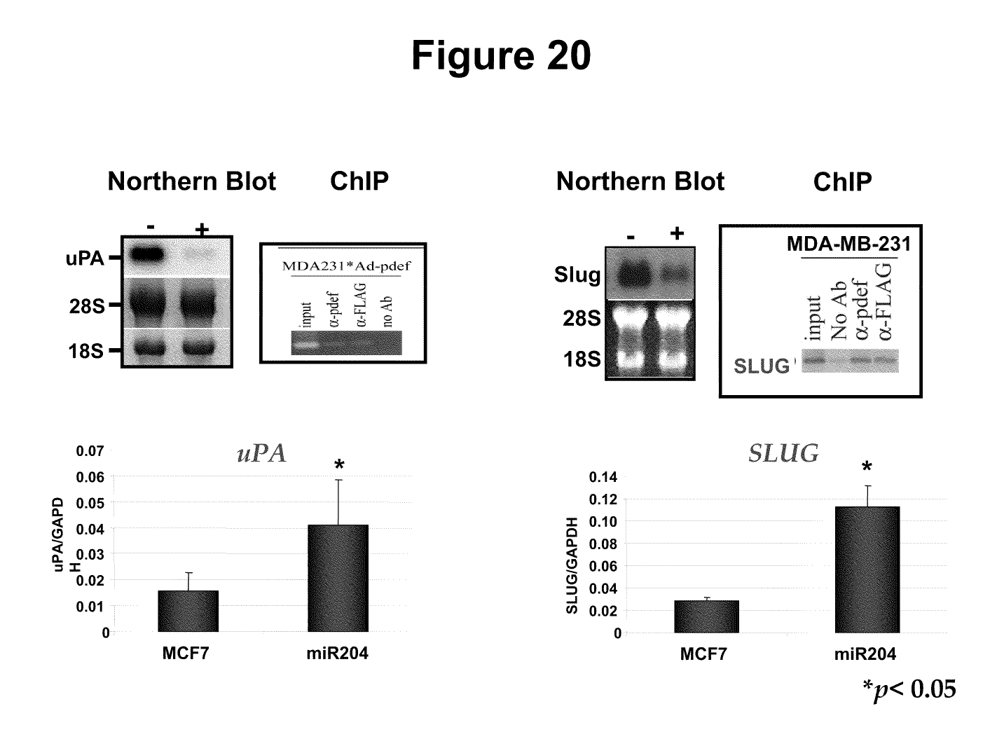

MicroRNAs have multiple targets and therefore the effects observed following miR-204 expression may be the result of the increased PDEF protein as well as non-PDEF related miR-204 affects. One way to evaluate these possibilities is to examine the phenotypes in cells in which a non-targeted PDEF is expressed. To do this, the ORF of PDEF was transfected into the miR-204 over-expressing cells and molecular and cellular phenotypic effects were measured. Quantitative real-time PCR analysis showed that exogenous PDEF expression in miR-204 over-expressing cells resulted in a decrease in both uPA and SLUG mRNA levels when compared to the vector alone (FIGS. 6A & B). In addition, these cells were less migratory and less invasive (FIGS. 6C & D). Thus, the miRNA mediated loss of PDEF was responsible for the observed changes. The collective data show that during breast cancer progression, elevated miR-204 reduces PDEF protein expression, and the resultant change in expression of PDEF-regulated genes and their downstream targets contributes to a more invasive phenotype.

miR-204 and miR-510 Levels are Elevated in Breast Tumor Samples

Based upon the impact of miR-204 and miR-510 on PDEF protein expression and PDEF dependent phenotypes, we evaluated the levels of miR-204 and miR-510 in RNA prepared from human breast tumor and matched non-tumor samples by quantitative real time PCR. Relative to that found in non-tumor tissue samples, the levels of miR-204 and miR-510 were found to be significantly elevated in all tumor samples. These data show that miR-204 and miR-510 function as ‘oncomiRs’ and further support the model that elevated expression of miRNAs contribute to the loss of PDEF protein expression during breast cancer progression. We have previously demonstrated that PDEF protein expression is reduced in invasive breast cancer specimens (3). PDEF expression was evaluated in 4 of the 5 samples evaluated for miRNA expression as they were available as formalin-fixed paraffin embedded blocks from the same patients (1335, 1412, 1420 & 1844). As shown in FIGS. 6C and 6D, PDEF protein is found predominantly in the nucleus of non-tumor epithelial cells. However, consistent with our previous findings, epithelial cells present in invasive ductal carcinomas show decreased protein expression. Taken together, these data show that miR-204 & -510 levels are elevated in invasive breast cancer and that there levels are inversely correlated to PDEF protein expression.

### Discussion

The rapidly evolving field of miRNAs has established close correlations between their altered expression and many types of cancer (30-32). However, the identification and validation of specific targets has been limited. This study identified two miRNAs (miR-204 and miR-510) that are involved in the negative regulation of PDEF mRNA translation and describes for the first time a mechanism that contributes to PDEF protein loss during breast cancer progression. Specifically, over-expression of miR-204 and -510 in MCF7 cells results in a dose-dependent loss of PDEF protein expression. Although ˜60% transfection efficiency was achieved in MCF7 cells, an apparently greater loss of PDEF protein expression was observed, perhaps due to a threshold level required for detection of protein by western blot analysis. A potential role for PDEF as a tumor suppressor has been demonstrated by studies showing reduced PDEF protein expression and/or loss by immunohistochemistry in invasive cancer (3), as well as the repression of survivin (33), uPA (3) and slug (Turner et al, manuscript in prep). Furthermore, recent studies showed PDEF protein expression inhibited xenograft tumor formation in vivo (33). Identifying the mechanisms of PDEF protein loss provides novel insight into the transcriptional networks that are active during metastatic progression. It is generally accepted that multiple mechanisms of regulation of any protein are active in normal cells and each of these represent pathways that can be altered during pathogenesis. Indeed, we show that regulatory elements are also present within the 5′UTR of PDEF, supporting a previous model that the 5′UTR of PDEF is involved in its post-transcriptional regulation in prostate cancer cells through inhibition of translation initiation (5). Interestingly, a recent study found that mRNA are repressed by engineering miRNA-binding sites in the 5′UTR as efficiently as in the 3′UTR, although no endogenous targets regulated in this manner have been found (34). In addition, both PDEF mRNA (4) and protein expression (Findlay et al., unpublished observations) are lost upon stimulation of cells with TGF-beta, a soluble growth factor able to induce EMT (35). The mechanism of TGF-beta dependent loss of PDEF mRNA and protein expression may include activation of inhibitory miRNAs, as a recent study illustrated a TGF-beta-induced EMT miRNA signature in human keratinocytes (36). Indeed, the EMT-like phenotype observed in MCF7 cells stably transfected with miR-204 or following transient expression of miR-510, supports the notion that growth factors, like TGF-beta, that induce EMT may play a role in the regulation of this and other miRNAs during breast cancer progression.

This and other studies have demonstrated the importance of PDEF regulation during breast cancer progression. We show that miR-204 and miR-510 levels are elevated in breast cancer compared to non-tumor tissue. Until now the expression levels of miR-204 and miR-510 have not been specifically examined in breast cancer specimens, although a high through-put microarray study has been performed for miRNA profiling on breast cancer biopsies (37). While miR-204 levels were not specifically highlighted in this study, the datasets show a significant elevation of miR-204 in those breast cancer biopsy samples with a tumor cell percentage of 70% or more when compared to the normal controls. miR-510 levels were not evaluated in this study. Of interest, the genomic locations of these two miRNAs have been associated with cancer. Amplification of a non-coding region mapped to chromosome 9q21 (miR-204) has been associated with prostate cancer (38). Furthermore, amplification of Xq27 (miR-510) occurs during the process of cell transformation and tumorigenesis using breast cancer as a model system and in sporadic breast cancer (39, 40). Future studies directed towards elucidating the mechanism of activation of these miRNAs will provide valuable insight into the complex pathways involved in metastatic breast cancer progression. This is exemplified by the recent study that identified the metastatic specific induction of miRNA-10b through the action of the transcription factor Twist (41), another known regulator of EMT, emphasizing the fundamental role for miRNAs in cancer progression and their representation as a new class of tumor suppressors and oncogenes.

The function of the majority of miRNAs is still currently unknown, although a plethora of predicted targets exist. We show here a validated target, PDEF, for two miRNAs, miR-204 and miR-510, as well as functional consequences of their interaction. We disclose that miR-204 and miR-510 are oncogenes and that their elevated expression contributes to the loss of PDEF protein expression during breast cancer progression.

### Experimental Results #2

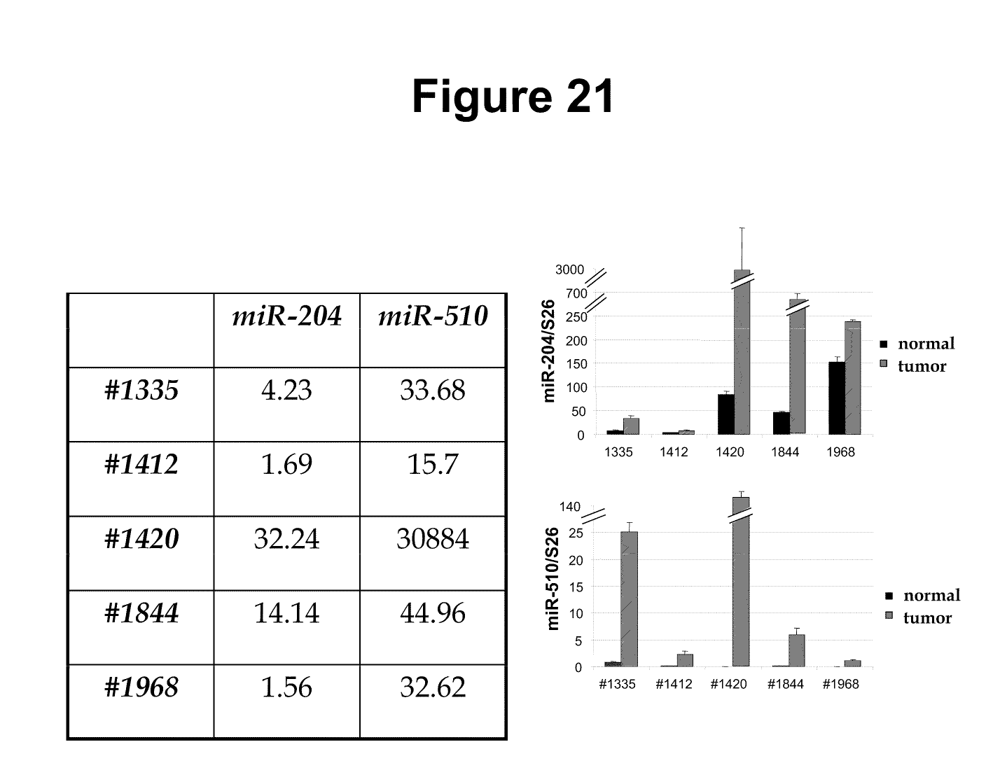

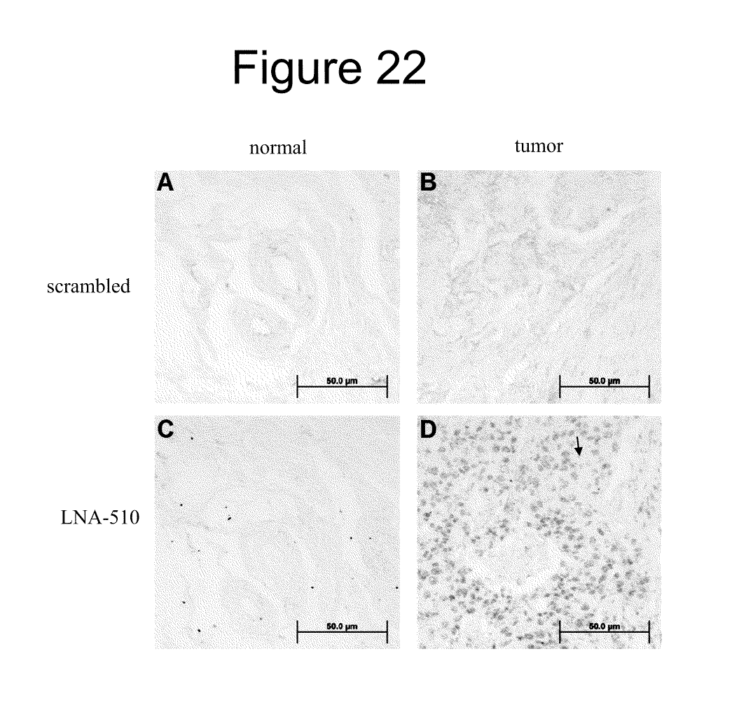

In Situ Hybridization (ISH) was performed with a locked nucleic acid (LNA) miR-510 probe (LNA-510) on a human breast tissue microarray (TMA). The human breast TMA consisted of duplicate cores of 35 cases of common types of malignant tumor and 13 cases of normal and non-malignant breast tissue. We observed positive staining in 14/35 (40%) malignant tumors and 0/13 normal and non-malignant breast tissue (data not shown). The use of a scrambled probe is recommended as a negative control and its importance is illustrated in FIG. 21 to distinguish background from positive staining The positive stain is a blue color and the nuclear counter-stain is red, therefore, the purple color of the stain in FIG. 21, shows that miR-510 is localized in the nucleus of the tumor cells (FIG. 22D).

Functional Consequences of microRNA-510 Expression in Non-Transformed Human Breast Cells: Over-Expression of miR510 and miR204 Promotes Transformation of Cells In Vitro

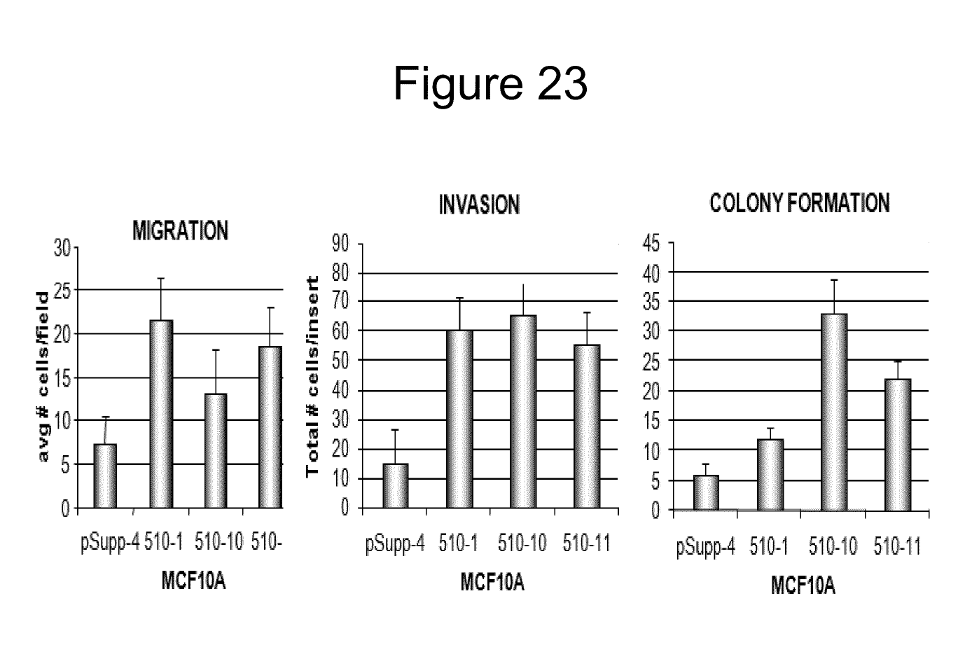

Based on the observations previously reported in the non-invasive breast cancer cell line MCF7, we hypothesized that miR-510 may have oncogenic potential. Therefore, to evaluate this and to further explore the role of miR-510 in breast cancer progression and its potential role in the induction of EMT, we generated stable clones in the non-transformed human breast cell line MCF10A. Our initial observations showed that miR-510 expression resulted in an altered morphology similar to that observed for MCF7 cells (data not shown). In addition, we observed an increase in the number of cells that were able to migrate, invade and form colonies in a clonogenic assay, processes that are required for tumor progression and metastasis (FIG. 23).

MicroRNA-510 modulates genes involved in tumor metastasis: Potential downstream RNAs modulated by miR expression—implicated in tumor metastasis. To further understand the more global role of miR-510 in the regulation of metastasis, we performed qPCR using a tumor metastasis superarray with RNA prepared from MCF10A cells stably expressing empty vector (pSupp-4) and three MCF10A miR-510 expressing stable clones (510-1, 510-10 and 510-11). We identified multiple genes that were modulated when compared to the empty vector control. We highlight those genes that were up/down-regulated at least 1.5-fold in all three stable clones analyzed (Table 1). None of the genes identified are listed as potential targets of miR-510 on the available databases. However, they may be indirect targets of miR-510 and provide valuable information as to the potential mechanism of miR-510 action in breast cells. Indeed, many of the genes identified as modulated in the PCR array are critical mediators of various processes involved in tumor metastasis. Therefore, the identification of direct targets of miR-510 reveal a central or key factor involved in the control of multiple mediators of metastasis and highlights the importance of the identification of such direct targets.

TABLE 3
Human Tumor Metastasis RT² Profiler ™ PCR Array. List of functional
gene groupings and the names of the genes that were modulated by the
stable over-expression of miR-510 in the non-transformed breast cell line
MCF10A. Up-regulated and down-regulated gene names in each functional
grouping are listed.

| FUNCTIONAL GENE GROUPINGS | UP-REGULATED | DOWN-REGULATED |
|---|---|---|
| Cell Adhesion Genes: | CD44, CDH11, | MCAM, MTSS1 |
| Cell to Cell Adhesion | FAT, SYK | |
| Transmembrane Receptors | CD44, ITGB3 | |
| Others Related to Adhesion | FN1 | |
| Extracellular Matrix Proteins: | MMP10, MMP9 | MMP11, MMP2 |
| Matrix Metalloproteinases | | TIMP3 |
| MMP Inhibitors | | |
| Cell Cycle Genes: | IL1B | TGFB1 |
| Regulation of the Cell Cycle | | MTSS1, TP53 |
| Negative Regulation of the Cell | | MYC, TP53 |
| Cycle | | |
| Cell Cycle Arrest and Checkpoint | | |
| Cell Growth and Proliferation | GNRH1, IL1B, | TGFB1 |
| Genes: | MDM2 | TNFSF10 |
| Negative Regulation of Cell | GNRH1 | CXCR4, EPHB2, |
| Proliferation | IL1B | FGFR4 |
| Growth Factors and Hormones | PLAUR | MYC |
| Cytokines and Chemokines | SYK | |
| Receptors | | |
| Other Genes Related to Growth | | |
| Apoptosis Genes: | IL1B | TIMP3, TNFSF10, |
| Induction of Apoptosis | | TP53 |
| Anti-apoptosis | | TGFB1 |
| Other Genes Related to Apoptosis | | CXCR4 |
| Transcription Factors and Regulators: | | ETV4, MYC, |
| Transcription Factors | | SMAD4, TP53 |
| Regulators | | SMAD2 |
| Other Genes Related to Metastasis: | CD82 | |

MicroRNA-510 is activated by the PI3K pathway: Identified upstream signaling pathway regulation of miR expression. Critical role for PI3K/Pten pathway.

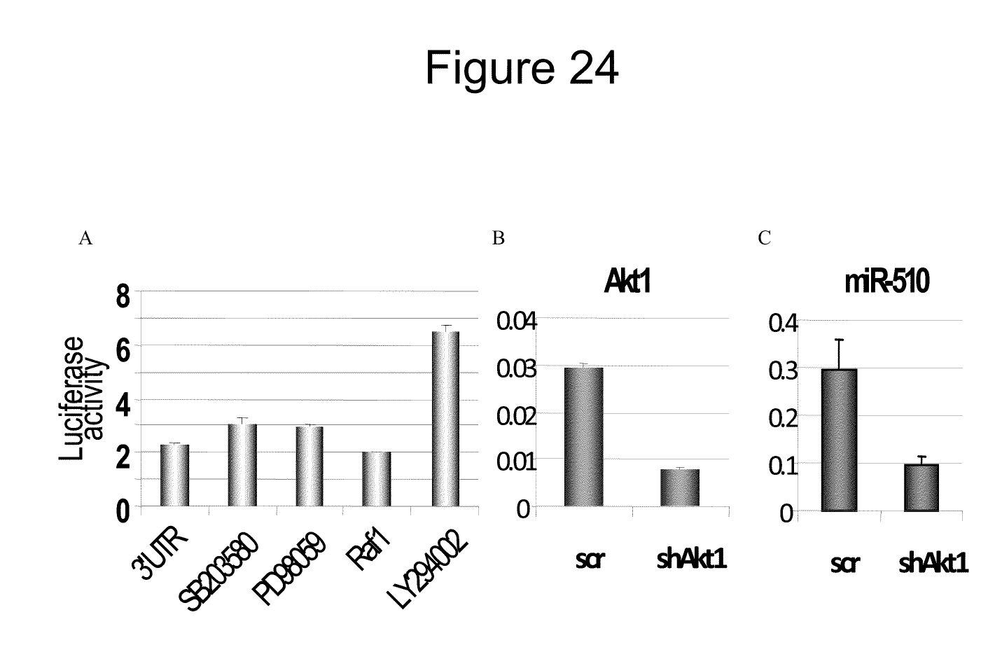

To understand the regulatory networks that are involved in the miR-510 induced tumor growth, progression and metastasis, it is important to identify the pathways that are involved in the direct regulation of miR-510. Our preliminary studies show that miR-510 is elevated in tumor cells when compared to normal cells. To address the mechanism of regulation of miR-510 in breast cancer cells, we used a reporter assay that has the 3′UTR of PDEF, a known direct target of miR-510, fused to a luciferase gene in order to indirectly evaluate the effect of specific kinase inhibition on miR-510 levels. A screen identified LY294002, a PI3K pathway inhibitor, as being the most potent activator of luciferase activity, a read out for the inhibition of miR-510 levels (FIG. 24A). In contrast, inhibition of p38, Erk1/2 or Raf kinases had little or no effect on the luciferase activity.

Upon identification of the potential involvement of the PI3K pathway in the regulation of miR-510, we utilized a lentiviral vector that contained a short hairpin to Akt1 to knockdown its expression. We transduced the human breast cancer cell line MDA-MB-175 VII with the lentiviral short hairpin vector to Akt1 (shAkt) or scrambled control (scr) and assessed the levels of Akt and miR-510 levels by qPCR. We observed a decrease in the mRNA levels of Akt, confirming successful transduction and knockdown. We also observed a decrease in the endogenous levels of miR-510 upon inhibition of Akt1 (FIG. 24C), demonstrating that the PI3K/Akt pathway is directly involved in the regulation of miR-510 expression.

MicroRNA-510 over-expression results in a positive feedback loop on the PI3K pathway.

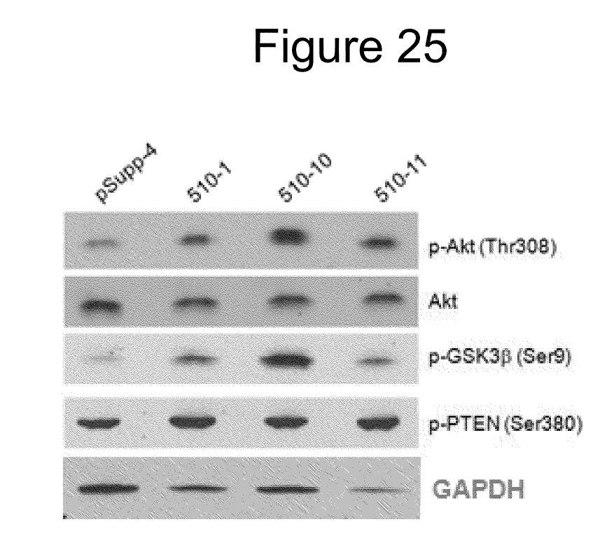

Our preliminary data show that miR-510 expression is positively regulated through the PI3K pathway (FIG. 24). In addition, we observed a number of downstream targets of this pathway (MDM2 & TP53) to be modulated upon miR-510 over-expression (Table 3). This led to the hypothesis that miR-510, once activated by PI3K, may result in a positive feedback loop resulting in enhanced activation of the PI3K pathway. We show that in our MCF10A miR-510 expressing clones (510-1, 510-10 and 510-11) that Akt is more active when compared to the vector-expressing control cells (p-Supp-4). This is illustrated by an elevation of phospho-Akt levels by western blot analysis (FIG. 25). Total Akt levels remain unchanged. To further evaluate the activation status of the Akt pathway we also examined the levels of phospho-GSK3β and phospho-PTEN and found them both to be elevated in the miR-510 over-expressing clones when compared to the vector control (FIG. 25). Phosphorylation of PTEN leads to a closed formation and less active form of the phosphatase. These data show that over-expression of miR-510 results in activation of the PI3K/Akt pathway and supports the hypothesis that PI3K and miR-510 function through a positive feedback loop.

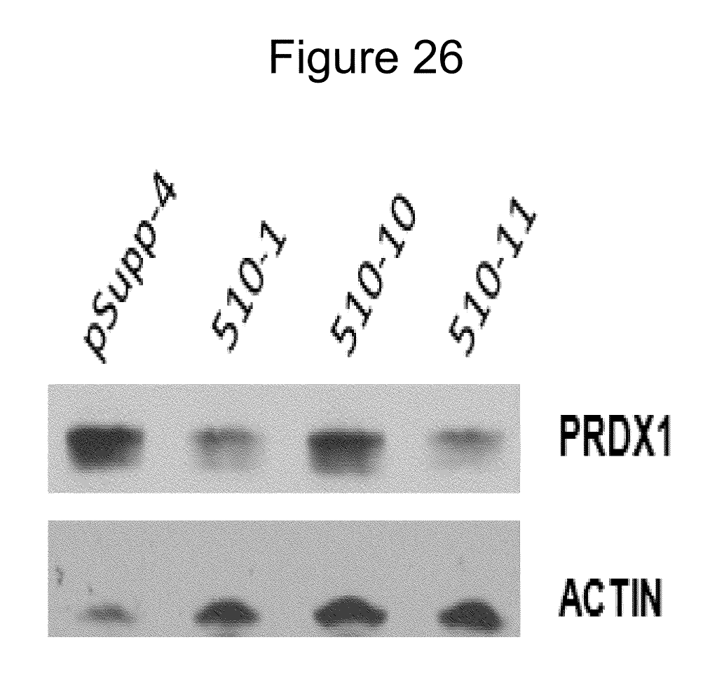

Interestingly, regulators and/or downstream effectors of the PI3K pathway are listed as predicted targets on the Miranda database (PML, PRDX1, GSK3β, CASP9), providing further evidence as to the potential importance of this pathway in the modulation of miR-510 and its downstream functional effects. Indeed, we show that PRDX1 protein expression is decreased in non-transformed immortalized breast cells stably over-expressing miR-510 (FIG. 26).

MicroRNA 510 Downstream Target Identification: Identified Novel Direct Targets for miR-510.

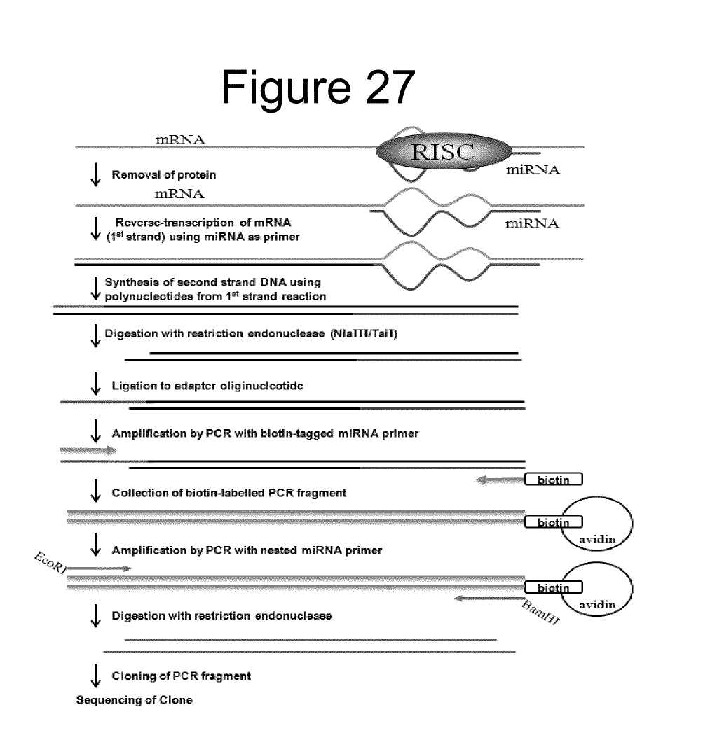

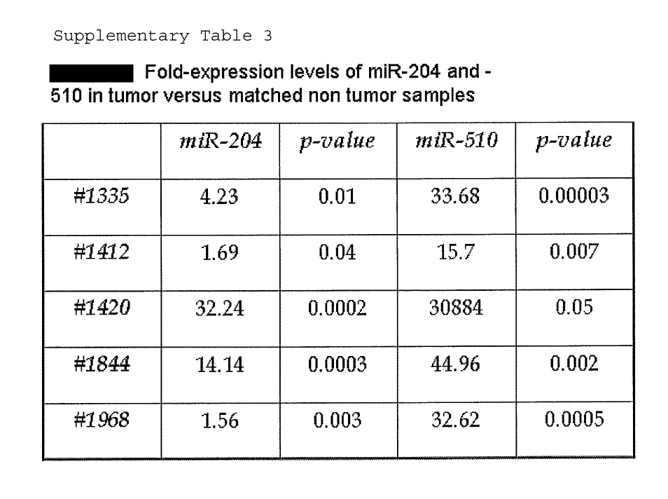

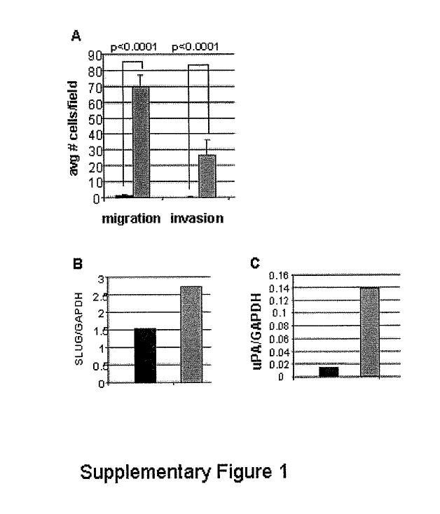

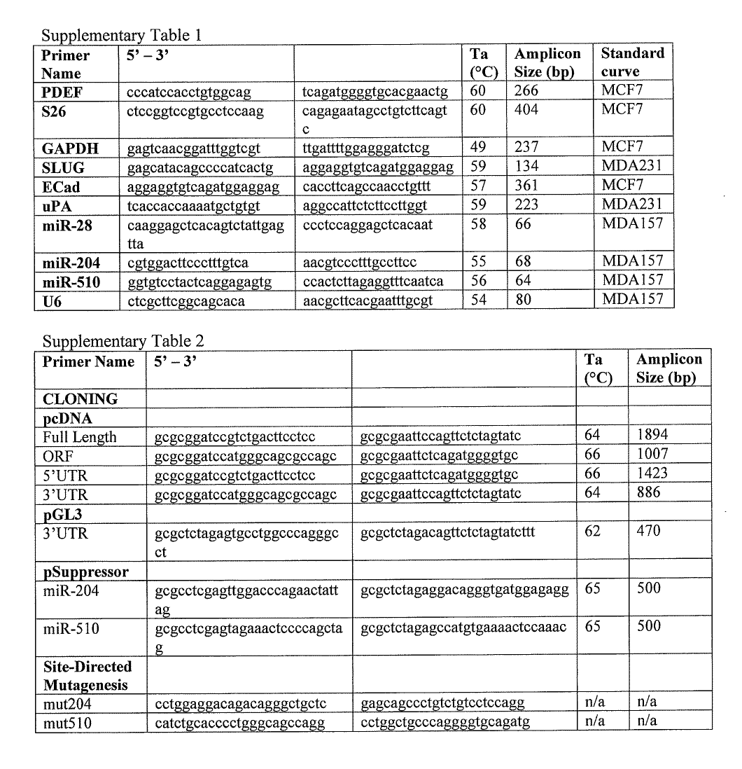

We utilized a novel biochemical approach to identify miR-510 target genes (FIG. 27). Briefly, this method isolates cDNA clones of target mRNAs that form base pairs in vivo with an endogenous miRNA of interest, in which the cDNAs are synthesized from the mRNAs using the miRNA as a reverse-transcription primer. The PCR fragments are then cloned into a vector containing LacZ sequence for blue/white screening and sequenced (FIG. 27).

We isolated 100 white colonies using this approach and performed colony lysis PCR with PDEF specific primers as a positive control and identified 3 PDEF containing inserts, showing that the approach successfully identified a validated downstream target of miR-510.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a microRNA measurement is disclosed and discussed and a number of modifications that can be made to the method are discussed, each and every combination and permutation of the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, BD, B-E, B-F, C-D, C-E, and C-F are specifically contemplated and should be considered disclosed from disclosure of A, B, and C; D, E, and F; and the example combination A D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Primers

Primers for use in the disclosed methods can be oligonucleotides having sequence complementary to the target sequence. This sequence is referred to as the complementary portion of the primer. The complementary portion of a primer can be any length that supports specific and stable hybridization between the primer and the target sequence. Generally this is 10 to 35 nucleotides long, but is preferably 16 to 24 nucleotides long. For whole genome amplification, it is preferred that the primers are from 12 to 60 nucleotides long.

B. Conformation Dependent Labels

Conformation dependent labels refer to all labels that produce a change in fluorescence intensity or wavelength based on a change in the form or conformation of the molecule or compound with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with the disclosed methods. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted, the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with the disclosed methods.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with the disclosed methods.

C. Detection Labels

To aid in detection and quantitation of microRNAs, detection labels can be incorporated into detection probes or detection molecules or directly incorporated into amplied nucleic acids. As used herein, a detection label is any molecule that can be associated with nucleic acid, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl(NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as Quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor

White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH-CH3, Diamino Phenyl 10 Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf15 Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, 25 Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-Nhydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4-tetrachlorofluorescein (TET), 5 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-30 Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or antidigoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2-dioxetane-3-2′-(5′-chloro)tricyclo [3.3.1.13,7]decane]-4-yl) phenylphosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect microRNAs or nucleic acid produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

D. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed microRNAs herein, is through defining the variants and derivatives in terms of homology to specific known sequences. This identity of particular sequences disclosed herein is also discussed elsewhere herein.

In general, variants of microRNAs herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods.

As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

E. Hybridization and Selective Hybridization

The term hybridization typically means a sequence driven interaction between at least two nucleic acid molecules, such as a primer or a probe and a microRNA. Sequence driven interaction means an interaction that occurs between two nucleotides or nucleotide analogs or nucleotide derivatives in a nucleotide specific manner. For example, G interacting with C or A interacting with T are sequence driven interactions.

Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize. Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, New 10 York, 1989; Kunkel et al. Methods Enzymol. 1987:154:367, 1987 which is herein incorporated by reference for material at least related to hybridization of nucleic acids).

A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their kd, or where only one of the nucleic acid molecules is 10 fold or 100 fold or 1000 fold or where one or both nucleic acid molecules are above their kd.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

F. Nucleic Acids

There are a variety of molecules disclosed herein that are nucleic acid based, including, for example, microRNAs. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment. So long as their relevant function is maintained, primers, probes, and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties.

Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 10 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-,S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH2)nO]mCH3, —O(CH2)nOCH3, -5-O—(CH2)nNH2, —O(CH2)nCH3, —O(CH2)n-ONH2, and —O(CH2)nON[(CH2)nCH3)]2, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2 CH3, ONO2, NO2, N3, NH2, 10 heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH2 and S. Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties. Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (amino ethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methylribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-Omethyl ribonucleotides. Such oligonucleotides and nucleic acids can be referred to as chimeric oligonucleotides and chimeric nucleic acids.

G. Solid Supports

Solid supports are solid-state substrates or supports with which molecules (such as probes) or other components used in, or produced by, the disclosed methods can be associated. Molecules can be associated with solid supports directly or indirectly. For example, probes can be bound to the surface of a solid support. An array is a solid support to which multiple probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination.

Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995).

A useful method of attaching oligonucleotides to solid-state substrates is described by Guo et al., Nucleic Acids Res. 22:5456-5465 (1994). Each of the components immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting and measuring microRNAs, the kit comprising amplification primers and detection probes. The kits also can contain enzymes and reaction solutions.

I. Mixtures

Disclosed are mixtures formed by performing or preparing to perform the disclosed method. For example, disclosed are mixtures comprising a body fluid and amplification primers, microRNA and amplification primers, and amplified microRNA and detection probes.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed.

The present disclosure contemplates these mixtures, obtained by the performance of the disclosed methods as well as mixtures containing any disclosed reagent, composition, or component, for example, disclosed herein.

J. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising microRNAs and a detection apparatus.

K. Data Structures and Computer Control

Disclosed are data structures used in, generated by, or generated from, the disclosed method. Data structures generally are any form of data, information, and/or objects collected, organized, stored, and/or embodied in a composition or medium. microRNA levels stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### REFERENCES

1. Seth A, Watson D K. ETS transcription factors and their emerging roles in human cancer. Eur J Cancer 2005; 41(16):2462-78.
2. Turner D P, Moussa O, Sauane M, Fisher P B, Watson D K. Prostate-derived ETS factor is a mediator of metastatic potential through the inhibition of migration and invasion in breast cancer. Cancer research 2007; 67(4):1618-25.
3. Feldman R J, Sementchenko V I, Gayed M, Fraig M M, Watson D K. Pdef expression in human breast cancer is correlated with invasive potential and altered gene expression. Cancer research 2003 ; 63 (15):4626-31.
4. Gu X, Zerbini L F, Otu H H, et al. Reduced PDEF expression increases invasion and expression of mesenchymal genes in prostate cancer cells. Cancer research 2007; 67(9):4219-26.
5. Nozawa M, Yomogida K, Kanno N, et al. Prostate-specific transcription factor hPSE is translated only in normal prostate epithelial cells. Cancer research 2000; 60(5):1348-52.
6. Gillanders W E, Mikhitarian K, Hebert R, et al. Molecular detection of micrometastatic breast cancer in histopathology-negative axillary lymph nodes correlates with traditional predictors of prognosis: an interim analysis of a prospective multi-institutional cohort study. Annals of surgery 2004; 239(6):828-37; discussion 37-40.
7. Mitas M, Mikhitarian K, Hoover L, et al. Prostate-Specific Ets (PSE) factor: a novel marker for detection of metastatic breast cancer in axillary lymph nodes. British journal of cancer 2002; 86(6):899-904.
8. Turner D P, Findlay V J, Moussa O, Watson D K. Defining ETS transcription regulatory networks and their contribution to breast cancer progression. Journal of cellular biochemistry 2007; 102(3):549-59.
9. Wiemer E A. The role of microRNAs in cancer: no small matter. Eur J Cancer 2007; 43(10):1529-44.
10. Bartel D P. MicroRNAs: genomics, biogenesis, mechanism, and function. Cell 2004; 116(2):281-97.
11. Rajewsky N. microRNA target predictions in animals. Nature genetics 2006; 38 Suppl:S8-13.
12. Shilo S, Roy S, Khanna S, Sen C K. MicroRNA in cutaneous wound healing: a new paradigm. DNA and cell biology 2007; 26(4):227-37.
13. Wang Y, Sticker H M, Gou D, Liu L. MicroRNA: past and present. Front Biosci 2007; 12:2316-29.
14. Chen K, Rajewsky N. The evolution of gene regulation by transcription factors and microRNAs. Nat Rev Genet. 2007; 8(2):93-103.
15. Hornstein E, Mansfield J H, Yekta S, et al. The microRNA miR-196 acts upstream of Hoxb8 and Shh in limb development. Nature 2005; 438(7068):671-4.
16. Li Y, Wang F, Lee J A, Gao F B. MicroRNA-9a ensures the precise specification of sensory organ precursors in Drosophila. Genes & development 2006; 20(20):2793-805.
17. Neilson J R, Zheng G X, Burge C B, Sharp P A. Dynamic regulation of miRNA expression in ordered stages of cellular development. Genes & development 2007; 21(5):578-89.
18. Esquela-Kerscher A, Slack F J. Oncomirs—microRNAs with a role in cancer. Nature reviews 2006; 6(4):259-69.
19. Garzon R, Fabbri M, Cimmino A, Calin G A, Croce C M. MicroRNA expression and function in cancer. Trends in molecular medicine 2006; 12(12):580-7.
20. Calin G A, Croce C M. MicroRNA signatures in human cancers. Nature reviews 2006; 6(11):857-66.
21. Calin G A, Ferracin M, Cimmino A, et al. A MicroRNA signature associated with prognosis and progression in chronic lymphocytic leukemia. The New England journal of medicine 2005; 353(17): 1793-801.
22. Volinia S, Calin G A, Liu C G, et al. A microRNA expression signature of human solid tumors defines cancer gene targets. Proceedings of the National Academy of Sciences of the United States of America 2006; 103(7):2257-61.
23. Yanaihara N, Caplen N, Bowman E, et al. Unique microRNA molecular profiles in lung cancer diagnosis and prognosis. Cancer cell 2006; 9(3):189-98.
24. Blenkiron C, Goldstein L D, Thorne N P, et al. MicroRNA expression profiling of human breast cancer identifies new markers of tumour subtype. Genome Biol 2007; 8 (10):R214.
25. Neve R M, Chin K, Fridlyand J, et al. A collection of breast cancer cell lines for the study of functionally distinct cancer subtypes. Cancer cell 2006; 10(6):515-27.
26. Brown K A, Aakre M E, Gorska A E, et al. Induction by transforming growth factor-beta1 of epithelial to mesenchymal transition is a rare event in vitro. Breast Cancer Res 2004; 6 (3):R215-31.
27. Gilles C, Polette M, Birembaut P, Brunner N, Thompson E W. Expression of c-ets-1 mRNA is associated with an invasive, EMT-derived phenotype in breast carcinoma cell lines. Clinical & experimental metastasis 1997; 15 (5):519-26.
28. Hajra K M, Chen D Y, Fearon E R. The SLUG zinc-finger protein represses E-cadherin in breast cancer. Cancer research 2002; 62(6):1613-8.
29. Peinado H, Portillo F, Cano A. Transcriptional regulation of cadherins during development and carcinogenesis. The International journal of developmental biology 2004; 48 (5-6):365-75.
30. He L, Thomson J M, Hemann M T, et al. A microRNA polycistron as a potential human oncogene. Nature 2005; 435(7043):828-33.
31. Lu J, Getz G, Miska E A, et al. MicroRNA expression profiles classify human cancers. Nature 2005; 435(7043):834-8.
32. O'Donnell K A, Wentzel E A, Zeller K I, Dang C V, Mendell J T. c-Myc-regulated microRNAs modulate E2F1 expression. Nature 2005; 435(7043):839-43.
33. Ghadersohi A, Pan D, Fayazi Z, Hicks D G, Winston J S, Li F. Prostate-derived Ets transcription factor (PDEF) downregulates survivin expression and inhibits breast cancer cell growth in vitro and xenograft tumor formation in vivo. Breast cancer research and treatment 2007; 102(1):19-30.
34. Lytle J R, Yario T A, Steitz J A. Target mRNAs are repressed as efficiently by microRNA-binding sites in the 5′ UTR as in the 3′ UTR. Proceedings of the National Academy of Sciences of the United States of America 2007; 104(23):9667-72.
35. Zavadil J, Bottinger E P. TGF-beta and epithelial-to-mesenchymal transitions. Oncogene 2005; 24(37):5764-74.
36. Zavadil J, Narasimhan M, Blumenberg M, Schneider R J. Transforming growth factor-beta and microRNA:mRNA regulatory networks in epithelial plasticity. Cells, tissues, organs 2007; 185 (1-3):157-61.
37. Mattie M D, Benz C C, Bowers J, et al. Optimized high-throughput microRNA expression profiling provides novel biomarker assessment of clinical prostate and breast cancer biopsies. Molecular cancer 2006; 5:24.
38. Bussemakers M J, van Bokhoven A, Verhaegh G W, et al. DD3: a new prostate-specific gene, highly overexpressed in prostate cancer. Cancer research 1999; 59(23):5975-9.
39. Balogh G A, Russo I H, Balsara B R, Russo J. Detection of chromosomal aberrations by comparative genomic hybridization during transformation of human breast epithelial cells in vitro. International journal of oncology 2006; 29(4):877-81.
40. Valladares A, Salamanca F, Madrigal-Bujaidar E, Arenas D. Identification of chromosomal changes with comparative genomic hybridization in sporadic breast cancer in Mexican women. Cancer genetics and cytogenetics 2004; 152(2):163-6.
41. Ma L, Teruya-Feldstein J, Weinberg R A. Tumour invasion and metastasis initiated by microRNA-10b in breast cancer. Nature 2007; 449(7163):682-8.
42. Oettgen P, Finger E, Sun Z, et al. PDEF, a novel prostate epithelium-specific ets transcription factor, interacts with the androgen receptor and activates prostate-specific antigen gene expression. The Journal of biological chemistry 2000; 275(2):1216-25.

We claim:

1. A method comprising detecting one or more target microRNAs in a tissue sample of a subject wherein the level of the one or more target microRNAs indicates the presence, severity, or stage of breast cancer in the subject.

2. The method of claim 1, wherein the one or more target microRNAs comprise one or more of miR-204 and miR-510.

3. The method of claim 1 wherein the tissue sample comprises a breast cancer tumor sample.

4. A method comprising detecting one or more target microRNAs in a tissue sample of a subject wherein the level of the one or more target microRNAs indicates the presence, severity, or stage of prostate cancer in the subject.

5. The method of claim 4, wherein the one or more target microRNAs comprise one or more of miR-204 and miR-510.

6. The method of claim 5 wherein the tissue sample comprises a prostate tumor sample.


SEQUENCE LISTING (40 sequences)

SEQ ID NO: 1 - RNA, 15 nt, Artificial Sequence
  chemically synthesized
ccuacuguuucccuu

SEQ ID NO: 2 - RNA, 21 nt, Artificial Sequence
  chemically synthesized
cacuaacggugagaggacuca

SEQ ID NO: 3 - DNA, 18 bp, Artificial Sequence
  chemically synthesized; primer for PDEF
cccatccacctgtggcag

SEQ ID NO: 4 - DNA, 21 bp, Artificial Sequence
  chemically synthesized; primer for PDEF
tcagatggggtgcacgaactg

SEQ ID NO: 5 - DNA, 20 bp, Artificial Sequence
  chemically synthesized; primer for S26
ctccggtccgtgcctccaag

SEQ ID NO: 6 - DNA, 23 bp, Artificial Sequence
  chemically synthesized; primer for S26
cagagaatagcctgtcttcagtc

SEQ ID NO: 7 - DNA, 20 bp, Artificial Sequence
  chemically synthesized; primer for GAPDH
gagtcaacggatttggtcgt

SEQ ID NO: 8 - DNA, 20 bp, Artificial Sequence
  chemically synthesized;  primer for GAPDH
ttgattttggagggatctcg

SEQ ID NO: 9 - DNA, 21 bp, Artificial Sequence
  chemically synthesized; primer for SLUG
gagcatacagccccatcactg

SEQ ID NO: 10 - DNA, 21 bp, Artificial Sequence
  chemically synthesized; primer for SLUG
aggaggtgtcagatggaggag

SEQ ID NO: 11 - DNA, 21 bp, Artificial Sequence
  chemically synthesized; primer for ECad
aggaggtgtcagatggaggag

SEQ ID NO: 12 - DNA, 20 bp, Artificial Sequence
  chemically synthesized; primer for ECad
caccttcagccaacctgttt

SEQ ID NO: 13 - DNA, 20 bp, Artificial Sequence
  chemically synthesized; primer for uPA
tcaccaccaaaatgctgtgt

SEQ ID NO: 14 - DNA, 20 bp, Artificial Sequence
  chemically synthesized; primer for uPA
aggccattctcttccttggt

SEQ ID NO: 15 - DNA, 26 bp, Artificial Sequence
  chemically synthesized; primer for miR-28
caaggagctcacagtctattgagtta

SEQ ID NO: 16 - DNA, 19 bp, Artificial Sequence
  chemically synthesized; primer for miR-28
ccctccaggagctcacaat

SEQ ID NO: 17 - DNA, 19 bp, Artificial Sequence
  chemically synthesized; primer miR-204
cgtggacttccctttgtca

SEQ ID NO: 18 - DNA, 18 bp, Artificial Sequence
  chemically synthesized; primer for miR-204
aacgtccctttgccttcc

SEQ ID NO: 19 - DNA, 21 bp, Artificial Sequence
  chemically synthesized; primer for miR-510
ggtgtcctactcaggagagtg

SEQ ID NO: 20 - DNA, 22 bp, Artificial Sequence
  chemically synthesized; primer miR-510
ccactcttagaggtttcaatca

SEQ ID NO: 21 - DNA, 17 bp, Artificial Sequence
  chemically synthesized; primer for U6
ctcgcttcggcagcaca

SEQ ID NO: 22 - DNA, 20 bp, Artificial Sequence
  chemically synthesized; primer for U6
aacgcttcacgaatttgcgt

SEQ ID NO: 23 - DNA, 24 bp, Artificial Sequence
  chemically synthesized; full length primer pcDNA
gcgcggatccgtctgacttcctcc

SEQ ID NO: 24 - DNA, 25 bp, Artificial Sequence
  chemically synthesized; full length primer pcDNA
gcgcgaattccagttctctagtatc

SEQ ID NO: 25 - DNA, 25 bp, Artificial Sequence
  chemically synthesized; ORF primer pcDNA
gcgcggatccatgggcagcgccagc

SEQ ID NO: 26 - DNA, 23 bp, Artificial Sequence
  chemically synthesized; ORF primer pcDNA
gcgcgaattctcagatggggtgc

SEQ ID NO: 27 - DNA, 24 bp, Artificial Sequence
  chemically synthesized; 5′UTR primer pcDNA
gcgcggatccgtctgacttcctcc

SEQ ID NO: 28 - DNA, 23 bp, Artificial Sequence
  chemically synthesized; 5′UTR primer pcDNA
gcgcgaattctcagatggggtgc

SEQ ID NO: 29 - DNA, 25 bp, Artificial Sequence
  chemically synthesized; 3′UTR primer pcDNA
gcgcggatccatgggcagcgccagc

SEQ ID NO: 30 - DNA, 25 bp, Artificial Sequence
  chemically synthesized; 3′UTR primer pcDNA
gcgcgaattccagttctctagtatc

SEQ ID NO: 31 - DNA, 28 bp, Artificial Sequence
  chemically synthesized; 3′UTR primer pGL3
gcgctctagagtgcctggcccagggcct

SEQ ID NO: 32 - DNA, 28 bp, Artificial Sequence
  chemically synthesized; 3′UTR primer pGL3
gcgctctagacagttctctagtatcttt

SEQ ID NO: 33 - DNA, 29 bp, Artificial Sequence
  chemically synthesized; miR-204 primer for pSuppressor
gcgcctcgagttggacccagaactattag

SEQ ID NO: 34 - DNA, 29 bp, Artificial Sequence
  chemically synthesized; miR-204 primer for pSuppressor
gcgctctagaggacagggtgatggagagg

SEQ ID NO: 35 - DNA, 28 bp, Artificial Sequence
  chemically synthesized; miR-510 primer for pSuppressor
gcgcctcgagtagaaactccccagctag

SEQ ID NO: 36 - DNA, 30 bp, Artificial Sequence
  chemically synthesized; miR-510 primer for pSuppressor
gcgctctagagccatgtgaaaactccaaac

SEQ ID NO: 37 - DNA, 24 bp, Artificial Sequence
  chemically synthesized; mut204 primer
cctggaggacagacagggctgctc

SEQ ID NO: 38 - DNA, 24 bp, Artificial Sequence
  chemically synthesized; mut204 primer
gagcagccctgtctgtcctccagg

SEQ ID NO: 39 - DNA, 24 bp, Artificial Sequence
  chemically synthesized; mut510 primer
catctgcacccctgggcagccagg

SEQ ID NO: 40 - DNA, 24 bp, Artificial Sequence
  chemically synthesized; mut510 primer
cctggctgcccaggggtgcagatg